(19) United States
(12) Patent Application Publication
(10) Pub. No.: US 2002/0182694 A1
(43) Pub. Date: Dec. 5, 2002

(54) Novel gene and polypeptide which opposes the Fas pathway

(76) Inventors: Paz Einat, Nes Zionna (IL); Hagar Kalinsky, Rishon-le-Zion (IL)

Correspondence Address: John P. White, Cooper & Dunham LLP, 1185 Avenue of the Americas, New York, NY, 10036, US

(21) Appl. No.: 10/160,669

(22) Filed: May 30, 2002

Related U.S. Application Data
(60) Provisional application No. 60/294,347, filed on May 30, 2001.

(51) Int. Cl. C12N 9/99; C07H 21/04; C12P 21/02; C12N 5/06

(52) U.S. Cl. 435/184; 435/69.2; 435/320.1; 435/325; 536/23.2

(57) ABSTRACT

This invention provides a purified polypeptide comprising consecutive amino acids the sequence of which extends from position 242 through position 380 of SEQ ID No:2. This invention also provides a purified polypeptide encoded by a polynucleotide having at least 30, preferably at least 50, more preferably at least 70, most preferably at least 100 consecutive nucleotides from position 1 to position 108 of SEQ ID NO:1. This invention further provides a purified polypeptide encoded by a polynucleotide having at least 30, preferably at least 50, more preferably at least 70, even more preferably at least 100, even more preferably at least 150, most preferably at least 200 consecutive nucleotides from position 340 to position 831 of SEQ ID NO:1. This invention further provides an isolated polynucleotide comprising consecutive nucleotides having a sequence as set forth in SEQ ID NO:1 and homologs or complements thereof, and this invention further provides an isolated polynucleotide comprising consecutive nucleotides having a sequence as set forth from position 93 through position 1232 of SEQ ID NO:1 and homologs or complements thereof or comprising consecutive nucleotides having a sequence incorporated in a plasmid designated pMLPD-606 Bac1, deposited under ATCC deposit No. PTA-4348, and homologs or complements thereof.

### PRIORITY

This application claims priority of U.S. Provisional application No. 60/294,347, filed May 30, 2001.

### BACKGROUND OF THE INVENTION

1. Field of the Invention

The present invention relates to Fas pathway genes. This application discloses a novel gene and polypeptide, termed the 606 gene and polypeptide. This gene/polypeptide is a negative regulator or modulator of the Fas apoptosis pathway. This gene/polypeptide is a viability gene/polypeptide and when its function is interrupted the cells become more sensitive to Fas-mediated apoptosis.

2. DESCRIPTION OF RELATED ART

Gene fragments and known genes involved in Fas-induced apoptosis are disclosed in U.S. Pat. No. 6,057,111 and in PCT application No. WO 98/21366, both assigned to QBI Enterprises Ltd. The PCT application discloses 36 fragments which may be involved in Fas-induced apoptosis. One of these fragments, SEQ ID No:18, corresponds to nucleotides 943 to 1125 of SEQ ID No:1 of the present application., which is only about 183 nucleotides out of the 1140 nucleotides in the amino acid encoding sequence of SEQ ID NO:1.

The sequence of the Genbank entry FLJ13287 hum-13643347 (the same FLJ13287 is represented by 10435245 and 13375888 that are almost identical to each other) from nucleotide 5 to nucleotide 2196 corresponds approximately to nucleotides 829 to 3034 of SEQ ID No 1 of the present application. The open reading frame of SEQ ID No:1 (the full length cDNA—see FIG. 1) is from nucleotide 93 to nucleotide 1232 of the polynucleotide depicted in FIG. 1.

An additional FLJ sequence was entered into Genbank on Feb 15, 2002.The sequence of this FLJ, FLJ23731 (gi: 18676878) from nucleotide 1 to nucleotide 3098 approximately corresponds to nucleotides 60 to 3024 of SEQ ID No 1. Note that there is a nucleotide missing between nucleotides 406 and 407 of FLJ23731. In SEQ ID No 1 the nucleotide at position 466 6 is G while in FLJ23731 the corresponding nucleotide at position 407 is C. Consequently, the protein encoded by FLJ23731 (protein accession BAB85046; gi: 18676879) is only 176 amino acids long and corresponds to amino acids 213 to 380 of the protein of SEQ ID No 2. The first 8 amino acids of BAB85046 do not match positions 205-212 of SEQ ID No:2 due to the missing nucleotide that caused an out of frame ATG to be wrongly regarded as the initial ATG; an additional inserted nucleotide in position 671 of the FLJ causes the frame to return to the original open reading frame, and thus amino acids 213 to 380 of SEQ ID No:2 are present in FLJ23731.

EST zb39c02 (Genbank) has two unconnected ends; r1 is in antisense orientation to nucleotides 702 through 1005 of SEQ ID No:1 of the present invention, is missing 3 nucleotides as compared with the stated positions of SEQ ID No:1, and has a mistake in one position; s1 corresponds to nucleotides 109 through 339 of SEQ ID No:1 of the present invention, and contains a mistake in one position.

### SUMMARY OF THE INVENTION

This application discloses a novel gene and polypeptide, termed the 606 gene and polypeptide. This gene/polypeptide is a negative regulator or modulator of the Fas apoptosis pathway. Inhibition of expression of this gene leads to the sensitization of cells to Fas mediated apoptosis.

### DESCRIPTION OF THE FIGURES

Other advantages of the present invention can be readily appreciated as the same becomes better understood by reference to the following detailed description when considered in connection with the accompanying drawings is wherein:

FIG. 1 depicts SEQ ID No:1, the nucleotide sequence of the 606 polynucleotide, of which positions 93 to 1232 encode the 606 polypeptide;

FIG. 2 depicts SEQ ID No:2, the amino acid sequence of the 606 polypeptide;

FIG. 3 depicts the translation of SEQ ID No:1 to SEQ ID No:2, wherein the amino acid translation is presented on the corresponding nucleotide positions;

FIG. 4 is a schematic representation of the Achilles Heel Method (AHM) by which the 606 fragment was identified;

FIG. 5 is a schematic representation of the AHM with a regulated anti-sense cDNA expression library;

FIG. 6 shows that CKI-7 sensitizes HeLa cells to FAS induced PCD;

FIGS. 7A-C show the effect of the antisense cDNA fragments identified by the AHM method on cell survival; A. shows that transfection of anti-sense bFGF sensitizes HeLa cells to Fas induced programmed cell death (PCD); B. shows the levels of expression of bFGF; C. shows the quantitation of the levels of the different bFGF forms;

FIGS. 8A-H show the validation of a gene identified by the AHM method as a negative regulator of Fas induced PCD in HeLa cells; A. shows that transfection of anti-sense Nrf2 sensitizes HeLa cells to Fas induced PCD; B. shows the levels of expression of Nrf2; C. shows that membrane permeable dominant negative Nrf2 polypeptide sensitizes HeLa cells to Fas induced PCD; D. shows that transfection of Nrf2 protects HeLa cells from Fas induced PCD; E. shows that Dicumarol sensitizes HeLa cells to Fas induced PCD as determined by the number of viable, trypan blue excluding cells; F. shows that Dicumarol sensitizes HeLa cells to Fas induced PCD as measured by the apoptotic index (calculated as the ratio of the number of apoptotic cells to the total number of cells as determined by DAPI staining); G. Shows that sulfinpyrazone sensitizes HeLa cells to Fas induced killing; and H. shows that N-acetyl cysteine protects HeLa cells from Fas induced PCD; and

FIG. 9 shows the results of a validation assay demonstrating that inhibition of the 606 gene sensitizes HeLa cells to Fas induced apoptosis.

### DETAILED DESCRIPTION OF THE INVENTION

The Achilles Heel Method (AHM) was used to identify various gene fragments as described herein below in Examples 1-3. In general three methods are available for the gene inactivation step: Genetic Suppressor Element (GSE) [Holzmeyer et al, 1992; Roninson et al, 1995; Gudkov et al, 1994], Random Homozygous Knock-Out (RHKO) [Li and Cohen, 1996], and Technical Knock Out (TKO) (Deiss and Kimchi, 1990) described herein above. Of these methods the TKO method is preferred as it is adaptable to the second step of the Achilles Heel Method (AHM).

In general, a wide variety of expression vectors can be used. This includes, but is not limited to, episomal vectors, retroviral vectors, adenoviral vectors and lentiviral vectors. The episomal vector is the preferable vector in the AHM method. Importantly, the episomal vector includes a selectable gene, such as the Hygromycin B resistance gene. This allows selection only of cells that contain the episomal vector since such cells are resistant to treatment with Hygromycin B while cells that do not have the episomal vector die.

In the AHM method cDNA fragments of a length range of approximately 200 to 600 bp (but not limited to this size range) derived from total cDNA, are introduced into episomal vector, thus making a special cDNA library in an episomal vector. We term such a fragment “Gene Inactivation Element” or “GIE” in short. Such a library is a collection of many (usually 100,000 to 1,000,000) different GIE episomal expression vectors. The episomal vector library, harbouring the GIEs, is then introduced into cultured cells. In this process many thousands of cells, up to few hundreds of thousand cells, each incorporate an episomal vector. The entire cell culture is then exposed to Hygromycin B and only cells that contain an episomal vector remain alive and keep multiplying. This resulting cell culture is termed “cell library”.

In each cell of the cell library a few copies of the episomal vector exist. However, it was found that in most cases within one cell all copies of the episomal vector have the same cDNA fragment. The cDNA fragment in the episomal vector, the GIE, is expressed, namely, transcribed into mRNA. The cDNA fragment, the GIE, in the episomal vector matches a part of an endogenous gene. When compared to the mRNA of the endogenous gene, GIE-derived mRNA can be either in antisense orientation or in the sense orientation. When in an antisense orientation the GIE-derived mRNA can inhibit the expression of the endogenous mRNA. When in the sense orientation the GIE-derived mRNA can encode a short polypeptide that can be a competitor of the matching endogenous protein. Such a short polypeptide is termed dominant negative peptide. Both posibilities lead to inhibition of the expression of the specific endogenous gene. Importantly, if in a given cell the expressed GIE inhibited a gene essential for general cell viability, such a cell will die or will stop multiplying and will quickly decrease in abundance. For example, if the mRNA encoding DNA polymerase II is inhibited, then the cell will not be able to multiply.

In the cell library every cell contains an episomal vector expressing a GIE. Thus, in each cell the function of a specific, and different, gene can be inhibited either by the inhibition of the mRNA by antisense mRNA or by inhibition of the protein by a dominant negative peptide.

Following the establishment of the cell library, an aliquot of the cells is exposed to a selection. That is, the cells are exposed to conditions requiring/activating a phenotype of interest. Such a phenotype includes, but is not limited to, activation of cell-death, causing growth arrrest, activation of contact inhibition, appearance or disappearance of specific markers (proteins or other distinctive chemical groups), activation or repression of specific promoters. The conditions requiring/activating the phenotye include, but are not limited to, treating cells with specific proteins, chemicals, antibodies, antisense oligonucleotides, exposing the cells to specific, special, growth conditions such as limiting oxygen levels, limiting amounts of nutrients in growth medium, and growing the cells to specific cell density. A reserved aliquot of the cell library is not exposed to the selection.

Following selection three types of cell behaviour are expected:

i. cells that retain the normal phenotype (or response), ii. cells that become resistant and show a “decreased” phenotype (e.g. do not show the phenotype or change their phenotype), and iii. cells which become sensitized to the selection conditions and show an enhanced phenotpe. In a cell death inducing selection, using a sub-lethal dose of the selection inducing condition that results in 20-50% cell death, the first type of behaviour will be normal rate of cell death, the second type of behaviour will be resistance to cell death causing such cells to increase in relative number in the population, and the third type of behaviour will be sensitization to cell death causing such cells to be more sensitive and to decrease in relative number in the population.

The second and third groups of cells that result from the selection are those in which the inactivation treatment caused inactivation of genes involved in the realization of the phenotype. The genes represented in the second group are positively involved, namely, they are required for the realization of the phenotype. The genes represented by the third group are negatively involved, namely, they normally opose the realization of the phenotype. The AHM method then provides for identifying the genes that belong to groups 2 and 3. This is accomplished by comparing the relative representation of the GIEs between the aliquot of selected cells to that in the aliquot of the reserved (i.e. untreated) cells.

As explained above, after selection the inactivation of specific genes causes some cells to increase in relative abundance and other cells to decrease in relative abundance. Since the inactivation events are caused by the GIEs expressed in the cells, then with the increase and decrease in cell abundance there is a matching increase and decrease in the GIEs found in the cells. In other words, the selection causes a differential representation of specific GIEs in the pool of cells obtained after the selection. The AHM method allows for the detection of the differentially represented GIEs and thus to detect the genes involved, positively and negatively, in the realization of the phenotype. All methods that can detect differential representation of nucleic acids are suitable.

This includes, but is not limited to, the techniques of differential display (Liang and Pardee, 1992; Liang et al., 1993), representational difference analysis (RDA), GEM-Gene Expression Microarrays (Schena et al., 1995); Aiello et al., 1994; Shen et al., 1995; Bauer et al., 1993;; 1995,; Braun et al., 1995, Hubank and Schatz, 1994; U.S. Pat. No. 5,545,531), high-density oligonucleotide arrays (Chee, M., Yang, R., Hubbell, E., Berno, A., Huang, X. C., Stern, D., Winkler, J., Lockhart, D. J., Morris, M. S. and Fodor, S.P.A Accessing Genetic Information with High-Density DNA Arrays Science 274:610-614, 1996; McGall, G. H., Labadie, J. Brock, P., Wallraff, G., Nguyen, T., Hinsberg, W. Light-Directed Synthesis of High-Density Oligonucleotide Arrays Using Semiconductor Photoresists. Proceedings of the National Academy of Sciences of the USA 93:13555-13560, 1996), suppressive subtraction hybridization (SSH), and direct sequencing (WO96/17957). The original gene fragment from which gene 606 was constructed and sequenced was found by subtraction.

Analysis by cDNA microarray is performed by deriving labeled probes from the GIE pools. Amplified GIEs from the unselected cells can be used to derive Cy3 labeled probe and amplified GIEs from the selected cells can be used to derive Cy5 labeled probe. The mixed Cy3 and Cy5 labeled probes are hybridized to a cDNA microarray. The microarrays preferentially have printed on them the actual GIE cDNA clones used in the screen. Following hybridization the differentially represented GIEs are detected as spots on the cDNA microarray (matching cDNA clones of GIEs) for which the ratio of Cy3 to Cy5 signal is eaither equal or greated than 2 or equal or lower than −2. In the embodiment described herein the clones for which the Cy3/Cy5 ratio is equal or greated than 2 represent the GIEs that inhibit negative modulators and thus directly identify the negative modulator genes. The clones for which which the Cy3/Cy5 ratio or equal or lower than −2 represent the GIEs that inhibit the positive modulators and thus directly identify the positive modulator genes. The identification of clone identity is done by sequencing the cDNA clone printed on the chip. Following sequencing, bioinformatics methods known in the art are used to compare the sequences to publicly available, and recognized, databases such as Genbank.

Analysis by subtraction is performed on the same two pools of GIEs. To detect the positive modulators, the pool of GIEs recovered from the selected cells is subtracted from those recovered from the unselected cells. This will result in the identification of GIEs enriched in the selected cells. To detect the negative modulators the pool of GIEs recovered from the unselected cells is subtracted from those recovered from the selected cells. This will result in the identification of GIEs depeleted in the selected cells. The preferred subtraction method used is the SSH method. The outcome of the method is a subtracted library. Clones are identified by sequencing followed by bioinformatics analysis as described above.

These cDNA, that are exact copies of the GIEs are then recloned into the vector used in the AHM screen and sequenced. The identified anti-sense expression vector of clone 606 was re-tested individually for the ability to inactivate the specific phenotype (see Example 5). With the sequence of the gene fragment identified, the sequence of the sense gene 606 was determined after a long and complicated process.; see Example 3.

AHM is used to identify of inhibitors of FAS induced apoptosis since the activation of the Fas pathway is relevant to several different pathologies. Activation of the Fas pathway is physiologically associated with both detrimental and with protective processes. For example, the activation of the Fas pathway is associated with liver damage in fulminate hepatitis and with immune mediated tissue destruction. Conversely, activation of the Fas pathway is required for prevention of autoimmunity (elimination of autoreactive T-cells) and suppression of tumorigenesis (Askew et al. 1991; Evan et al. 1992; Shi et al. 1992; Wagner et al. 1994). Thus, it is clinically beneficial to generate tools for enhancing (stimulating) or inhibiting the Fas pathway depending on the pathology of interest. The identification of inhibitors/stimulators of the Fas pathway can be used for both of these purposes. Certain genes identified by the Fas AHM screen, such as gene 606, are putative survival agents. As such, over-expression of these genes are predicted to prevent killing, and thus the 606 polypeptide can be used for clinical benefit in situations where cell survival is desired such as organ failure, neurodegeneration etc. Accelerating FAS induced programmed cell death (PCD) can ameliorate auto-immunity and enhance tumor suppression, thus, pharmacological inhibition of the FAS pathway inhibitors can be translated into significant clinical benefit, as they accelerate killing.

Additionally, gene/polypeptide 606 can then be used as targets to develop inhibitors (cofactors) which activate the pathway. For example, gene 606 might inhibit killing induced by chemotherapeutics or may be a general survival gene. Such genes can be important for the deleterious growth properties of cancer cells. Inhibition of such genes sensitizes tumors to chemotherapeutics. Inhibitors of this inhibitor gene have utility in treating cancer patients.

The invention provides a method for producing a nucleic acid molecule involved in the Fas pathway by nucleic acid sequence homology using a nucleic acid probe, the sequence of which is derived from the nucleic acid sequence encoding the nucleic acid molecule. The method of making and using the probes are known in the art e.g. see references cited in EXAMPLES section under the heading GENERAL METHODS.

The invention provides a method for producing a nucleic acid molecule involved in the Fas pathway which includes the use of the polymerase chain reaction and oligonucleotide primers, the sequence of which is derived from the nucleic acid sequence encoding the nucleic acid molecule. The method of making the primers and using them in the polymerase chain reaction are known in the art e.g. see references cited in EXAMPLES section under the heading GENERAL METHODS.

The Fas pathway genes are of two types. One type of genes oppose (cause inhibition of) Fas-induced killing; gene 606 is such a gene. A compound which stimulates such a Fas pathway gene is a compound which causes an increase in the amount and/or the activity of gene product; this compound renders the cells more resistant to Fas-induced apoptosis. A compound which inhibits such a Fas pathway gene is a compound which causes an decrease in the amount and/or the activity of gene product; this compound renders the cells more sensitive to Fas-induced apoptosis.

The second type of genes cause stimulation of Fas-induced killing. A compound which stimulates such a Fas pathway gene is a compound which causes an increase in the amount and/or the activity of gene product; this compound renders the cells more sensitive to Fas-induced apoptosis. A compound which inhibits such a Fas pathway gene is a compound which causes an decrease in the amount and/or the activity of gene product; this compound renders the cells more resistant to Fas-induced apoptosis.

PCD can also take place via pathways other than Fas, and gene 606 described here can also function in such pathways.

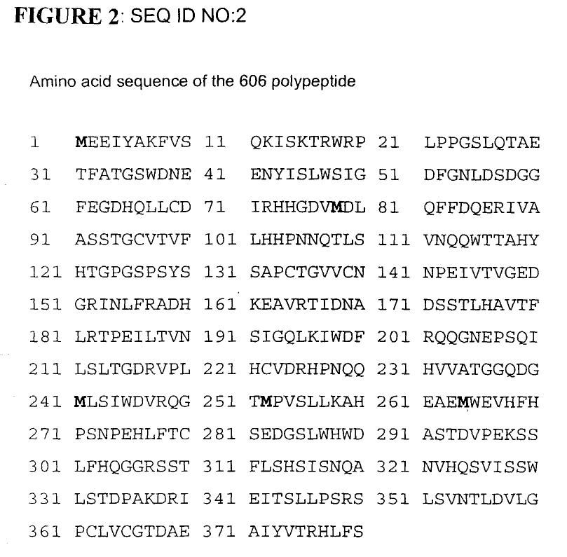

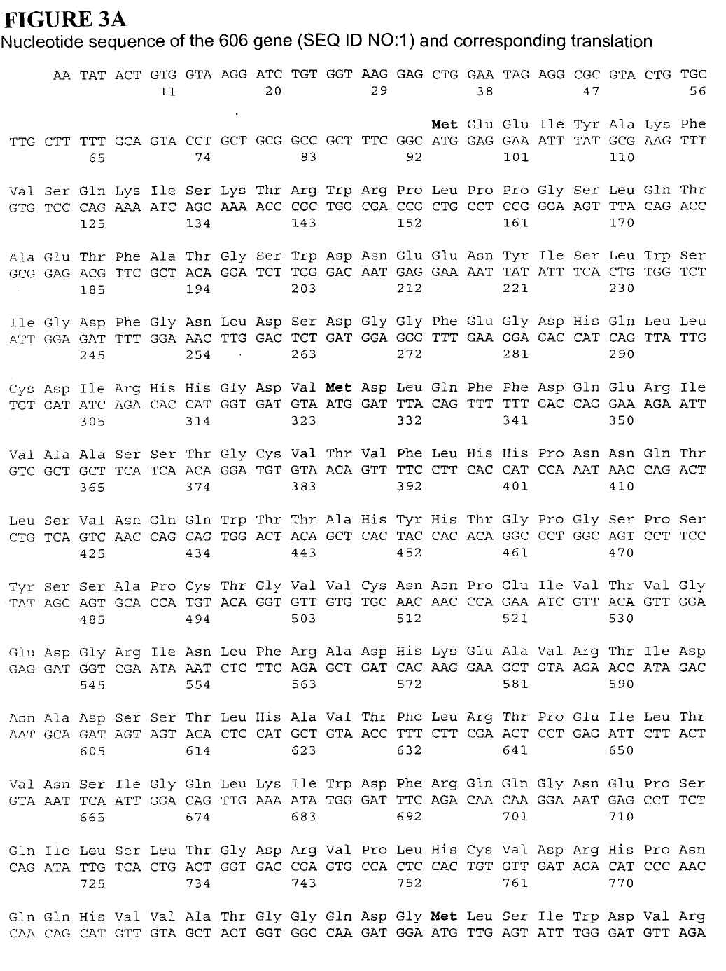

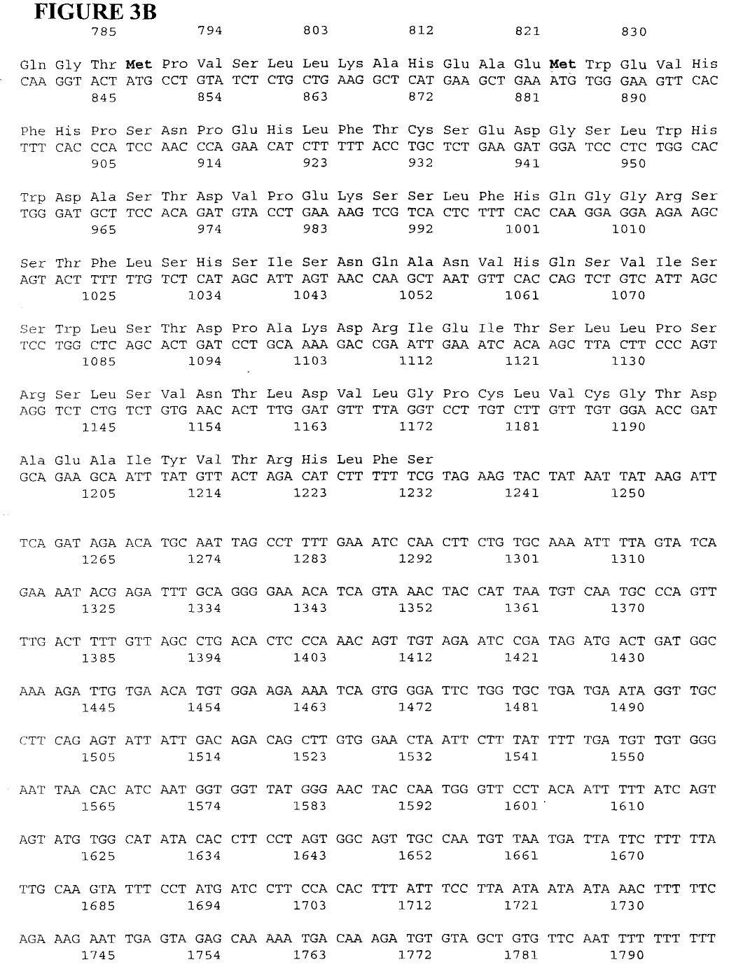

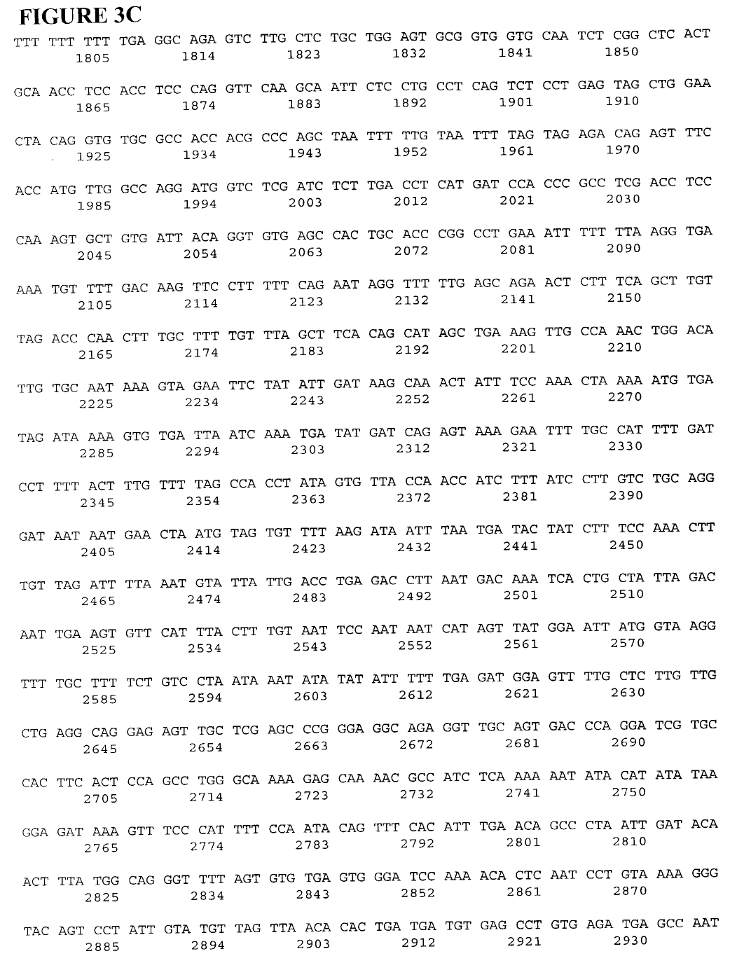

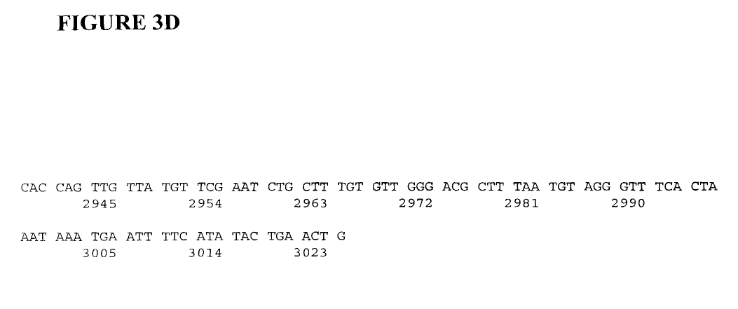

One embodiment of the present invention is directed to a purified 606 polypeptide comprising consecutive amino acids the sequence of which extends from position 242 through position 380 of SEQ ID No:2 (see FIG. 2), from position 241 through position 380 of SEQ ID No:2, from position 79 through position 380 of SEQ ID No:2, from position 78 through position 380 of SEQ ID No:2, from position 2 through position 380 of SEQ ID No:2, or from position 1 through position 380 of SEQ ID No:2, and homologs thereof. The polypeptide may be encoded by consecutive nucleotides present in a plasmid designated pMLPD-606 Bac1, deposited under ATCC deposit No. PTA-4348. Polypeptides of the present invention may also include polypeptides comprising consecutive amino acids from additional positions of SEQ ID No:2.

Another embodiment of the present invention provides for the above purified polypeptide having the biological activity of opposing Fas-mediated apoptosis, and homologs thereof having at least 70% homology, preferably at least about 80% homology, more preferably at least about 90% homology, most preferably at least about 95% homology, and retaining the biological activity of opposing Fas-mediated apoptosis.

The biological activity of opposing Fas-mediated apoptosis can be assayed and measured according to the methods described herein in examples 1, 2 and 4 (for example with an assay based on Fas-antibody), and is also termed “606 biological activity” in the instant application. The polypeptide of the present invention and fragments thereof, as depicted in SEQ ID No:2 (see FIG. 2), is understood to possess a multitude of biological activities, among them the biological activity of opposing Fas-mediated apoptosis. Any polypeptides described as dominant negative peptides to the polypeptide of the present invention are understood to inhibit 606 biological activity, and are also considered a part of the present invention.

In addition, the present invention provides for the above purified polypeptide (as specified according to the positions in SEQ ID No:2) having the inhibitory activity of a dominant negative peptide to the aforementioned polypeptide, and thus blocking the biological activity of opposing Fas-mediated apoptosis, and homologs thereof having at least 70% homology, preferably at least about 80% homology, more preferably at least about 90% homology, most preferably at least about 95% homology, and retaining the inhibitory activity of blocking opposition to Fas-mediated apoptosis.

A further embodiment of the present invention is directed to a purified polypeptide encoded by a polynucleotide having at least 30, preferably at least 50 more preferably at least 70, most preferably at least 100 consecutive nucleotides from position 1 to position 108 of SEQ ID NO:1, or a purified polypeptide encoded by a polynucleotide having at least 30, preferably at least 50 more preferably at least 70, even more preferably at least 100, even more preferably at least 150, most preferably at least 200 consecutive nucleotides from position 340 to position 831 of SEQ ID NO:1, or from position 1 to position 3024 of SEQ ID NO:1, provided that said polypeptide or polynucleotide encoding it has not been prior disclosed in any one of: FLJ13287, protein ID No BAB14536.1, ESTzB39cO2 or PCT patent application publication No. WO 98/21366.

Particular fragments of the 606 polypeptide depicted in SEQ ID No:2 include amino acids 1-50, 51-100,101-150, 151-200 and 201-251 of the sequence shown in FIG. 2. Further particular fragments of the 606 polypeptide depicted in SEQ ID No:2 include amino acids 25-74, 75-124, 125-174, 175224 and 225-274 of the sequence shown in FIG. 2. Additional particular fragments of the 606 polypeptide depicted in SEQ ID No:2 include fragments of at least 5, preferably at least 10, more preferably at least 15, even more preferably at least 20, even more preferably at least 25 and most preferably at least 30 consecutive amino acids of the sequence shown in FIG. 2, in so far as they were not prior disclosed in Genebank protein ID No. BAB14536.1.

The present invention is also directed to an isolated polynucleotide comprising consecutive nucleotides having a sequence which encodes any one of the above polypeptides, which may be incorporated in a plasmid designated pMLPD-606 Bac1, deposited under ATCC designation No. PTA-4348, and may comprise a strand of full length cDNA. In addition, the present invention is directed to a polynucleotide which is an antisense polynucleotide to the polynucleotide which encodes any one of the above polypeptides.

A further ambodiment of the present invention provides an isolated polynucleotide comprising consecutive nucleotides having a sequence as set forth in SEQ ID NO:1, a polynucleotide having a sequence that differs from SEQ ID No.1 due to the degeneracy of the genetic code or, or a polynucleotide which homologous or complementary thereto. Polynucleotides which differ from such a polynucleotide due to the existence of introns in a genomic gene sequence are also considered an embodiment of the invention.

A further ambodiment of the present invention provides an isolated polynucleotide comprising consecutive nucleotides having a sequence as set forth from position 93 to position 1232 of SEQ ID No:1, or an isolated polynucleotide comprising consecutive nucleotides having a sequence as incorporated in a plasmid designated pMLPD-606 Bac1, deposited under ATCC deposit No. PTA-4348, and homologs or complements thereof.

More in particular, the invention further comprehends isolated and/or purified polynucleotides (nucleic acid molecules) and isolated and/or purified polypeptides having at least about 70%, preferably at least about 75%; more preferably at least about 80%, even more preferably at least about 90%, most preferably at least about 95% homology to the polynucleotides and polypeptides disclosed herein. The invention also comprehends that these homologous polynucleotides and polypeptides can be used in the same fashion as the herein or aforementioned polynucleotides and polypeptides. Nucleotide sequence homology can be determined using the “Align” program of Myers and Miller, ((1988) CABIOS 4:11-17) and available at NCBI. Alternatively or additionally, the term “homology” for instance, with respect to a nucleotide or amino acid sequence, can indicate a quantitative measure of homology between two sequences. The percent sequence homology can be calculated as (Nref-Ndif)*100/Nref, wherein Ndif is the total number of non-identical residues in the two sequences when aligned and wherein Nref is the number of residues in one of the sequences. Hence, the DNA sequence AGTCAGTC has a sequence similarity of 75% to AATCAATC (Nref=8; Ndif =2).

Alternatively or additionally, “homology” with respect to sequences can refer to the number of positions with identical nucleotides or amino acid residues divided by the number of nucleotides or amino acid residues in the shorter of the two sequences wherein alignment of the two sequences can be determined in accordance with the Wilbur and Lipman algorithm ((1983) Proc. Natl. Acad. Sci. USA 80:726), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc., Calif.). When RNA sequences are said to be similar, or to have a degree of sequence identity or homology with DNA sequences, thymidine (T) in the DNA sequence is considered equal to uracil (U) in the RNA sequence. RNA sequences within the scope of the invention can be derived from DNA sequences or their complements, by substituting thymidine (T) in the DNA sequence with uracil (U).

Additionally or alternatively, amino acid sequence similarity or homology can be determined, for instance, using the BlastP program (Altschul et al. Nucl. Acids Res. 25:3389-3402) and available at NCBI. The following references provide algorithms for comparing the relative identity or homology of amino acid residues of two proteins, and additionally, or alternatively, with respect to the foregoing, the teachings in these references can be used for determining percent homology : Smith et al. (1981) Adv. Appl. Math. 2:482-489; Smith et al. (1983) Nucl. Acids Res. 11:2205-2220; Devereux et al. (1984) Nucl. Acids Res. 12:387-395; Feng et al. (1987) J. Molec. Evol. 25:351-360; Higgins et al. (1989) CABIOS 5:151-153; and Thompson et al. (1994) Nucl. Acids Res. 22:4673-480. Polynucleotide sequences that are complementary to any of the sequences or fragments encompassed by the present invention discussed above are also considered to be part of the present invention. Whenever any of the sequences discussed above are produced in a cell, the complementary sequence is concomitantly produced and, thus, the complementary sequence can also be used as a probe for the same diagnostic purposes.

An additional embodiment of the present invention provides an antibody directed to an epitope on any one of the above polypeptides, and an antibody binding specifically to any one of the above polypeptides.

By the term “antibody” as used in the present invention is meant both poly- and mono-clonal complete antibodies as well as fragments thereof, such as Fab, F(ab′)₂, and Fv, which are capable of binding the epitopic determinant. These antibody fragments retain the ability to selectively bind with its antigen or receptor and are exemplified as follows, inter alia:

(1) Fab, the fragment which contains a monovalent antigen-binding fragment of an antibody molecule can be produced by digestion of whole antibody with the enzyme papain to yield a light chain and a portion of the heavy chain;

(2) (Fab′)₂, the fragment of the antibody that can be obtained by treating whole antibody with the enzyme pepsin without subsequent reduction; F(ab′₂) is a dimer of two Fab fragments held together by two disulfide bonds;

(3) Fv, defined as a genetically engineered fragment containing the variable region of the light chain and the variable region of the heavy chain expressed as two chains; and

(4) Single chain antibody (SCA), defined as a genetically engineered molecule containing the variable region of the light chain and the variable region of the heavy chain linked by a suitable polypeptide linker as a genetically fused single chain molecule.

By the term “epitope” as used in this invention is meant an antigenic determinant on an antigen to which the antibody binds. Epitopic determinants usually consist of chemically active surface groupings of molecules such as amino acids or sugar side chains and usually have specific three dimensional structural characteristics, as well as specific charge characteristics.

The present invention further provides for pharmaceutical compositions comprising any one of the polypeptides, polynucleotides or antibodies of the present invention, and for vectors comprising any of the polynucleotides of the present invention, and compositions comprising said vectors.

A further embodiment of the present invention concerns a method of treating a tumor or an auto-immune disease in a subject which comprises administering to the subject a therapeutically effective amount of a chemical composition which inhibits the 606 biological activity of the polypeptide of the present invention. The chemical composition may comprise an antisense polynucleotide or a polypeptide acting as a dominant negative peptide, according to the present invention and as described above.

The term “tumor” as used herein is intended to include, but is not limited to, cancers of various types including carcinoma, lymphoma, melanoma and leukemia, inter alia.

An additional embodiment of the present invention concerns a method of treating a degenerative disease in a subject which comprises administering to the subject a therapeutically effective amount of a chemical composition which enhances or stimulates the 606 biological activity of the polypeptide of the present invention. The chemical composition may comprise a polynucleotide, a vector which comprises a polynucleotide or a polypeptide having 606 biological activity, according to the present invention and as described above.

The term “degenerative disease” as used herein, is intended to include, but is not limited to, degenerative disease of the liver, especially fulminate hepatitis, as well as stroke, Parkinson's disease, Epilepsy, Depression, ALS (Amyotrophic lateral sclerosis), Alzheimer's disease, Huntington's disease and any other disease-induced dementia (such as HIV induced dementia for example). In addition, “degenerative disease” is understood to include such conditions as hypertension, hypertensive cerebral vascular disease, rupture of an aneurysm, a constriction or obstruction of a blood vessel—as occurs in the case of a thrombus or embolus, angioma, blood dyscrasias, any form of compromised cardiac function including cardiac arrest or failure, systemic hypotension, cardiac arrest, cardiogenic shock, septic shock, spinal cord trauma, head trauma, seizure, bleeding from a tumor.

A further embodiment of the present invention provides for the use of any one of the polypeptides, polynucleotides or antibodies of the present invention, as described above, in the preparation of a medicament.

An additional aspect of the present invention concerns a method for identifying a chemical compound which modulates Fas-mediated apoptosis which comprises:

(a) contacting a cell expressing a polypeptide of the present invention with the compound; and

(b) determining the ability of the compound to modulate Fas-mediated apoptosis of the cell as compared to a control.

A control can be cells treated with the same chemical compounds, but not expressing the gene, and/or a control can be cells expressing the gene but not treated with the chemical compounds.

The term “modulation” is understood to include partial or full inhibition, stimulation and enhancement.

The cell in step (a) may be genetically engineered to express the polypeptide. “Genetically engineered” in this context includes transduced, transfected, and infected with a polynucleotide or vector which causes the cell to express the polypeptide.

An additional aspect of the present invention provides a method of screening a plurality of chemical compounds not known to modulate Fas-mediated apoptosis to identify a compound which modulates Fas-mediated apoptosis which comprises:

(a) contacting cells expressing a polypeptide of the present invention with the plurality of chemical compounds not known to modulate Fas-mediated apoptosis;

(b) determining whether the Fas-mediated apoptosis of the cell is modulated in the presence of the compounds, as compared to a control; and if so

(c) separately determining whether the modulation of Fas-mediated apoptosis is increased by each compound included in the plurality of compounds, so as to thereby identify the compound which modulates Fas-mediated apoptosis.

The cell in step (a) may be genetically engineered to express the polypeptide.

Additionally, the present invention provides a non cell-based method for identifying a compound which modulates Fas-mediated apoptosis comprising:

(a) measuring the interaction of a polypeptide or a polynucleotide of the present invention with an interactor;

(b) contacting the polypeptide or polynucleotide with said compound; and

(c) determining whether the interaction between the polypeptide or the polynucleotide and said interactor is affected by said compound.

The the interaction of step (a) may occur through a WD40 domain on the polypeptide or the polynucleotide.

The term “interactor” refers to any molecule or compound, whether organic or inorganic, which can form an interaction with the specified molecule, be it inter alia a binding interaction, electrostatic interaction, ionic interaction or any other interaction in which the interactor affects the specified molecule in any way.

The present invention also provides for a method of preparation of a pharmaceutical composition which comprises:

(a) identifying a chemical compound which modulates Fas-mediated apoptosis according to one of the methods described above, and;

(b) admixing said compound or a chemical homolog or analog thereof with a pharmaceutically acceptable carrier.

Methods by which compounds which modulate Fas-mediated apoptosis can be identified, validated and used in the preparation of pharmaceutical compositions are detailed in example 4 below.

An additional embodiment of the present invention concerns a kit for identifying a compound which modulates Fas-mediated apoptosis which comprises:

A polypeptide and/or a polynucleotide of the present invention; an interactor with which the polypeptide and/or polynucleotide interacts;

means for measuring the interaction of the polypeptide and/or the polynucleotide with the interactor;

means of contacting the polypeptide and/or the polynucleotide with said compound; and

means of determining whether the interaction between the polypeptide and/or polunucleotide and the interactor is affected by said compound.

Chemical Compounds

The compounds to be administered comprise inter alia small chemical molecules, antibodies, antisense oligonucleotides, antisense DNA or RNA molecules, proteins, polypeptides and peptides including peptido-mimetics and dominant negatives, and expression vectors

Negative dominant (or dominant negative) peptide or polypeptide refers to a polypeptide encoded by a partial cDNA sequence that encodes for a part of a protein (see Herskowitz, 1987). This peptide can have a different function from the protein from which it was derived. It can interact with the full protein and inhibit its activity or it can interact with other proteins and inhibit their activity in response to the full protein. Negative dominant means that the peptide is able to overcome the natural proteins and fully inhibit their activity to give the cell a different characteristic like resistance or sensitization to killing. For therapeutic intervention either the peptide itself is delivered as the active ingredient of a pharmaceutical composition or the cDNA can be delivered to the cell utilizing the same methods as for antisense delivery.

Antisense therapeutic construct can be delivered to the cells and can be rendered nuclease resistant as is known in the art [Agrawal, 1996; Calabretta, et al, 1996; Crooke, 1995; Felgner, 1997; Gewirtz, 1993; Hanania, et al 1995; Lefebvre-d'Hellencourt et al, 1995; Lev-Lehman et al., 1997; Loke et al, 1989; Wagner et al., 1996; Wagner, 1994; Radhakrishnan et al., 1990.] As shown in the Examples below, many fragments of sequences of genes have been identified. An antisense construct of these sequences delivered to a cell reduces a gene product (gene inactivation) and thereby provides stimulation or opposition of Fas-induced apoptosis, depending on the gene involved. These antisense constructs can be used therapeutically.

Delivery of Chemical Compounds

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

The compound of the present invention can be administered in various ways. It should be noted that it can be administered as the compound or as pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, nontoxic solid or liquid fillers, diluents or encapsulating material not reacting with the active ingredients of the invention.

It is noted that humans are treated generally longer than the mice or other experimental animals exemplified herein which treatment has a length proportional to the length of the disease process and drug effectiveness. The doses can be single doses or multiple doses over a period of several days, but single doses are preferred.

The doses can be single doses or multiple doses over a period of several days. The treatment generally has a length proportional to the length of the disease process and drug effectiveness and the patient species being treated.

When administering the compound of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques which deliver it orally or intravenously and retain the biological activity are preferred. In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used. The quantities to be administered vary for the patient being treated and vary from about 100 ng/kg of body weight to 100 mg/kg of body weight per day and preferably are from 10 μg/kg to 10 mg/kg per day.

Gene Therapy:

By gene therapy as used herein refers to the transfer of genetic material (e.g DNA or RNA) of interest into a host to treat or prevent a genetic or acquired disease or condition phenotype. The genetic material of interest encodes a product (e.g. a protein, polypeptide, peptide, functional RNA, antisense) the production of which in vivo is desired. For example, the genetic material of interest can encode a hormone, receptor, enzyme, polypeptide or peptide of therapeutic value. Alternatively, the genetic material of interest may encode a suicide gene. For a review see, in general, the text “Gene Therapy” (Advances in Pharmacology 40, Academic Press, 1997).

Two basic approaches to gene therapy have evolved: (1) ex vivo and (2) in vivo gene therapy. In ex vivo gene therapy cells are removed from a patient, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject rather the genetic material to be transferred is introduced into the cells of the recipient organism in situ, that is within the recipient. In an alternative embodiment, if the host gene is defective, the gene is repaired in situ [Culver, 1998]. These genetically altered cells have been shown to express the transfected genetic material in situ.

The gene expression vehicle is capable of delivery/transfer of heterologous nucleic acid into a host cell. The expression vehicle can include elements to control targeting, expression and transcription of the nucleic acid in a cell selective manner as is known in the art. It should be noted that often the 5′UTR and/or 3′UTR of the gene can be replaced by the 5′UTR and/or 3′UTR of the expression vehicle. Therefore as used herein the expression vehicle can, as needed, not include the 5′UTR and/or 3′UTR of the actual gene to be transferred and only include the specific amino acid coding region.

The expression vehicle can include a promotor for controlling transcription of the heterologous material and can be either a constitutive or inducible promotor to allow selective transcription. Enhancers that can be required to obtain necessary transcription levels can optionally be included. Enhancers are generally any non-translated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al (1986) and include, for example, stable or transient transfection, lipofection, electroporation and infection with recombinant viral vectors. In addition, see U.S. Pat. No. 4,866,042 for vectors involving the central nervous system and also U. S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector for introducing and expressing recombinant sequences is the adenovirus derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most cancers of epithelial origin as well as others. This vector as well as others that exhibit similar desired functions can be used to treat a mixed population of cells and can include, for example, an in vitro or ex vivo culture of cells, a tissue or a human subject.

Additional features can be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotic. Such protection ensures that if, for example, mutations arise that produce altered forms of the viral vector or recombinant sequence, cellular transformation can not occur.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods of the invention depends on desired cell type to be targeted and is known to those skilled in the art. For example, if breast cancer is to be treated then a vector specific for such epithelial cells are used. Likewise, if diseases or pathological conditions of the hematopoietic system are to be treated, then a viral vector that is specific for blood cells and their precursors, preferably for the specific type of hematopoietic cell, is used. Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed do not contain a genome and therefore cannot proceed through subsequent rounds of infection. The specific type of vector depends upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, and administration can also be performed by, for example, intravenous or subcutaneous injection into the subject. Injection of the viral vectors into a spinal fluid can also be used as a mode of administration, especially in the case of neuro-degenerative diseases. Following injection, the viral vectors circulate until they recognize host cells with the appropriate target specificity for infection.

An alternate mode of administration can be by direct inoculation locally at the site of the disease or pathological condition or by inoculation into the vascular system supplying the site with nutrients or into the spinal fluid. Local administration is advantageous because there is no dilution effect and, therefore, a smaller dose is required to achieve expression in a majority of the targeted cells. Additionally, local inoculation can alleviate the targeting requirement required with other forms of administration since a vector can be used that infects all cells in the inoculated area. If expression is desired in only a specific subset of cells within the inoculated area, then promoter and regulatory elements that are specific for the desired subset can be used to accomplish this goal. Such non-targeting vectors can be, for example, viral vectors, viral genome, plasmids, phagemids and the like. Transfection vehicles such as liposomes can also be used to introduce the non-viral vectors described above into recipient cells within the inoculated area. Such transfection vehicles are known by one skilled within the art.

The above discussion provides a factual basis for identification of genes that are involved in the Fas pathway, and their use in drug discovery and treatment. The methods used and, in particular, the identification of the 606 gene are shown in the following non-limiting examples and accompanying figures.

### EXAMPLES

General Methods:

General Methods in Molecular Biology:

Standard molecular biology techniques known in the art and not specifically described were generally followed as in Sambrook et al., Molecular Cloning: A Laboratory Manual, Cold Springs Harbor Laboratory, New York (1989, 1992), and in Ausubel et al., Current Protocols in Molecular Biology, John Wiley and Sons, Baltimore, Md. (1989).

Polymerase chain reaction (PCR) was carried out generally as in PCR Protocols: A Guide To Methods And Applications, Academic Press, San Diego, Calif. (1990). Reactions and manipulations involving other nucleic acid techniques, unless stated otherwise, were performed as generally described in Sambrook et al., 1989, Molecular Cloning: A Laboratory Manual, Cold Spring Harbor Laboratory Press, and methodology as set forth in U.S. Pat. Nos. 4,666,828; 4,683,202; 4,801,531; 5,192,659 and 5,272,057 and incorporated herein by reference.

Recombinant Protein Purification is undertaken as generally set forth in Marshak et al, “Strategies for Protein Purification and Characterization. A laboratory course manual.” CSHL Press, 1996 unless otherwise specified.

Vectors are constructed containing the cDNA of the present invention by those skilled in the art and should contain all expression elements necessary to achieve the desired transcription of the sequences (see below in specific methods for a more detailed description). Other beneficial characteristics can also be contained within the vectors such as mechanisms for recovery of the nucleic acids in a different form. Phagemids are a specific example of such beneficial vectors because they can be used either as plasmids or as bacteriophage vectors. Examples of other vectors include viruses such as bacteriophages, baculoviruses and retroviruses, DNA viruses, cosmids, plasmids, liposomes and other recombination vectors. The vectors can also contain elements for use in either procaryotic or eucaryotic host systems. One of ordinary skill in the art knows which host systems are compatible with a particular vector.

The vectors are introduced into cells or tissues by any one of a variety of known methods within the art (calcium phosphate transfection; electroporation; lipofection; protoplast fusion; polybrene transfection). The host cell can be any eucaryotic and procaryotic cells, which can be transformed with the vector and which supports the production of the enzyme. Methods for transformation can be found generally described in Sambrook et al., Molecular Cloning: A Laboratory Manual, Cold Springs Harbor Laboratory, New York (1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995) and Gilboa, et al. (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

General Methods in Immunology:

Standard methods in immunology known in the art and not specifically described were generally followed as in Stites et al.(eds), Basic and Clinical Immunology (8th Edition), Appleton & Lange, Norwalk, Conn. (1994) and Mishell and Shiigi (eds), Selected Methods in Cellular Immunology, W. H. Freeman and Co., New York (1980).

Immunoassays:

In general, ELISAs are the preferred immunoassays employed to assess a specimen. ELISA assays are well known to those skilled in the art. Both polyclonal and monoclonal antibodies can be used in the assays. Where appropriate other immunoassays, such as radioimmunoassays (RIA) can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, Molecular Cloning: A Laboratory Manual, Cold Springs Harbor, N.Y., 1989.

### Polyclonal and Monoclonal Antibody Production

Antibody Production:

Antibodies can be either monoclonal or polyclonal. Conveniently, the antibodies can be prepared against a synthetic peptide based on the sequence, or prepared recombinantly by cloning techniques or the natural gene product and/or portions thereof can be isolated and used as the immunogen. Such proteins or peptides can be used to produce antibodies by standard antibody production technology well known to those skilled in the art as described generally in Harlow and Lane, Antibodies: A Laboratory Manual, Cold Spring Harbor Laboratory, Cold Spring Harbor, N.Y., 1988 and Borrebaeck, Antibody Engineering—A Practical Guide, W. H. Freeman and Co., 1992.

For producing polyclonal antibodies a host, such as a rabbit or goat, is immunized with the protein or peptide, generally with an adjuvant and, if necessary, coupled to a carrier; antibodies to the protein are collected from the sera.

For producing monoclonal antibodies the technique involves hyperimmunization of an appropriate donor with the protein or peptide fragment, generally a mouse, and isolation of splenic antibody producing cells. These cells are fused to a cell having immortality, such as a myeloma cell, to provide a fused cell hybrid which has immortality and secretes the required antibody. The cells are then cultured, in bulk, and the monoclonal antibodies harvested from the culture media for use.

The monoclonal antibodies as defined include antibodies derived from one species (such as murine, rabbit, goat, rat, human, etc.) as well as antibodies derived from two (or more) species, such as chimeric and humanized is antibodies.

The antibody can be bound to a solid support substrate or conjugated with a detectable moiety or be both bound and conjugated as is well known in the art. For a general discussion of conjugation of fluorescent or enzymatic moieties see Johnstone & Thorpe, Immunochemistry in Practice, Blackwell Scientific Publications, Oxford, 1982. The binding of antibodies to a solid support substrate is also well known in the art. (see for a general discussion Harlow & Lane Antibodies: A Laboratory Manual, Cold Spring Harbor Laboratory Publications, New York, 1988 and Borrebaeck, Antibody Engineering —A Practical Guide, W. H. Freeman and Co., 1992) The detectable moieties contemplated with the present invention can include, but are not limited to, fluorescent, metallic, enzymatic and radioactive markers such as biotin, gold, ferritin, alkaline phosphatase, â-galactosidase, peroxidase, urease, fluorescein, rhodamine, tritium, ¹⁴C and iodination.

Completely human antibodies are particularly desirable for therapeutic treatment of human patients. Human antibodies can be made by a variety of methods known in the art including phage display methods using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Additional information regarding all types of antibodies, including humanized antibodies, human antibodies and antibody fragments can be found in WO 01/05998, which is incorporated herein by reference in its entirety.

### Specific Methods

Construction of GIE Expression Vector:

The EBV based episomal vector [Deiss et al, 1991] consists of DNA segments that are necessary for the episomal maintenance of the episome both in bacteria (E. coli) and in human cells (this include an origin of replication and a trans-acting factor (EBNA-1). The episome also includes genes encoding resistance markers for selection either in bacteria or in human cells. Finally the vector contains a transcription cassette. The EBV episomal vector contains a RNA Polymerase II promoter and or enhancer driving the transcription of a synthetic transcript containing a set of cloning sites, a splice donor and acceptor site and a polyadenylation signal, followed by a second set of enhancers. This vector can be efficiently shuttled from animal cells to bacteria and vice versa. One procedure that allows for rapid shuttling is using the method of Hirt [1967] to extract episomal vectors from animal cells and using this preparation to transform E.coli. Applicants have observed that, on average, cells transfected with a library cloned into the vector contain few copies of only one species of expressing vector.

The specific choice of promoters and enhancers are dependent on the exact selection condition and the cell line used. This must be empirically determined for each selection condition as is known to those skilled in the art.

The EBV vector can also contain an inducible expressed promoter such that the expression of the anti-sense library can be inducibly expressed by a specific inducer. This allows additional flexibility in designing selection protocols.

Construction of GIE cDNA Library

In the prefered embodiment of AHM, the GIE library mostly contain cDNAs in the antisense orientation. There are several methods available for construct ion of directional cDNA libraries. Any of these methods can be sufficient since they result in the production of a directionally identified cDNA library and the practitioners can use the method they are most familiar with. The directional cDNA is then cloned into the expression cassette in the anti-sense orientation. A method that can be used is detailed in Deiss et al, 1991. Briefly, it consists of making cDNA by the method of Gubler and Hoffman [1983] and making the cDNA directional by the method of Meisner et al. [1987]. It should be noted that in most of these methods the majority of cDNA fragments will be in the antisense orientation but there will always be some percentage of cDNA fragments that will be in the sense orientation.

The mRNA is extracted from cells that have been cultured under a variety of conditions that mimic the actual selection conditions. This is designed to ensure that the library includes all the messages that are expressed in the target cell under selection conditions. RNA is prepared at time points that can contain messages that are always present as well as messages that are induced by the selection procedure. This is achieved by extracting RNA previous to the selection and at times during the selection. The various pools of RNA are then mixed together so that all possible RNA molecules are present [Deiss and Kimchi, 1991].

An alternative method that can be used consists of deriving a library of genomic DNA fragments cloned into the expression cassette. This method can generate all possible messages, since all the transcribed messages are derived from genomic DNA (with the exception of RNA edited messages; this actually includes mitochondrial DNA as well). The directionality can be lost so the library can be only half anti-sense. Since the sense fragments are less likely to frequently encode full length proteins or have biological activity the anti-sense fragments can still likely produce the most frequent biological effects. The genomic fragments are produced by restriction enzyme cleavage of genomic DNA. It is necessary to produce only one library per species, since with the exception of the B and T cell receptors, genomic DNA does not differ in different cell types at least in mammals (again erythrocytes or any cells that lack nuclei are an exception).

In the case of the genomic library it is necessary to determine whether any expressed fragments express a sense or an anti-sense message. This is done by using the insert as a strand specific probe both from the expressed and non-expressed strand in a Northern analysis. This indicates if the expressed fragment is sense or anti-sense in relation to the endogenously expressed gene. Some sequences match, some do not match, genes already deposited in the various databases. In the case where the identified gene matches the sequence of a gene already in a database this information can then enable the determination if the insert is a sense or anti-sense insert.

Transfection of the GIE Library.

There are a large variety of methods to introduce DNA into cells of cell lines and cell cultures. The most efficient method for each selection can be determined empirically based on experience and the known relative efficiency of each method.

The method selected must both efficiently deliver the DNA into cells and not affect the biological responses that can be selected following the transfection. Viral vector system can also be used and this can entail producing infectious virus and infecting the target cells. Applicant has found electroporation to be an efficient method, but other methods can be used as are known in the art.

Identification of Differentially Represented GIEs

The methods of identifying the differentially represented GIEs in a preferred embodiment can include the methods described in Braun [1996] and as described in Diatchenko et al.[1996]. These methods include a PCR amplification of subtracted populations. Appropriate restriction sites are included in the expression vector so that following PCR amplification of the cDNA inserts, the inserts are flanked by appropriate restriction sites. Restriction digestion is then used to produce templates that are useful for these techniques.

Another method that is used is the GEM gene expression microarray as described in Schena et al. [1995]. In this technique, PCR fragments dereived from a set of specific plasmids that contain the fragments, (in this case the GIE cDNA inserts contained in the episomal vectors or other DNAs as appropriate) are fixed to a glass template and this is hybridized with two fluorescently labeled probes. In this specific embodiment, the probes are derived from the pools of episomal vectors rescued from the cell library after selection and from the unselected reserved cell library aliquote. The GIEs found in the rescued episomal vectors are amplified using primers matching the common sequences flanking the inserts. In a prefered embodiment of this approach one of the primers have in its end (the 5′ region of the primer) the T7 RNA polymerase promoter sequence. The amplified GIE inserts are then used as templates for the production of a labeled probe as follows. A T7 RNA polymerase reaction is performed to produce RNA copies of the amplified GIE insets. This RNA is then reverse transcribed in the presence of flourescent nucleotides to produce labeled single stranded cDNA. GIEs rescued from the untreated, reserved, cell aliquote are used to prepare a probe labeled by one type of fluorescent label such as Cy3. GIEs rescued from the cell library after selection are used to prepare a probe labeled by another type of fluorescent label such as Cy5. The two probes are mixed together and hybridized to the cDNA microarray, Following hybridization the cDNA microarray is scanned, once for the Cy3 fluorescence and once for the Cy5 fluorescence. Next the ratio of Cy3/Cy5 signals is determined. Two sets of spots on the cDNA microarray are derived. The first set include all spots that show a value of Cy3/Cy5 that is equal or greater then +2. This set include all GIEs that were depleted after selection. In the FAS apoptosis screen this group represens endogenous putative “viability” genes oposing FAS induced apoptosis. The second set include all spots that show a value of Cy3/Cy5 that is equal or lower than −2. This set include all GIEs that were enriched after selection. In the FAS apoptosis screen this group represens endogenous putative “pro-apoptotic” genes supporting FAS induced apoptosis. Since all spots have known corresponding plasmid clones, the gene identity of the spots can easily be determined. The plasmid clones are sequenced and the sequence of the inserts is compared to gene databases.

Another option for making probes for CDNA microarray analysis uses total RNA derived from the cell libraries. Poly-A+mRNA derived from the total RNA is reverse-transcribed using a common complementary primer to the transcribed 3′UTR region of the episomal vector. This region exists after the GIE insertion location in the episomal vector and is common to all GIEs in the library. Thus, the reverse transcription reaction will reverse transcribe all GIE-derived mRNA but not endogenous mRNA. The labeling with fluorescent nucleotides can take place during this reverse transcription reaction. Another option for producing labeled probes from these templates is to use a common complementary primer (as described above) that have a T7 RNA polymerase promoter sequence in its 5′ region. The single-stranded cDNA produced is transformed into double-stranded cDNA that becomes a template for a T7 RNA polymerase reaction. The RNA produced from this reaction is then labeled with the fluorescent nucleotides exactly as described above.

Generation of Efficient GIE Antisense Inhibitors

In addition to assaying expression cassettes where most of the transcripts are directionally cloned in the antisense orientation, another strategy employed in the present invention is to generate randomly primed cDNA and cleave the cDNA with two restriction enzymes X and Y, and clone the resulting mixture into two different expression cassettes. In the first cassette, site X can transcriptionally precede Y and in the second cassette site Y can transcriptionally precede X. In this arrangement, the cDNA is divided into sections that can have different abilities to serve as an efficient antisense inhibitor. The strongest differential signal is likely to be produced by the fragment that is the most efficient antisense inhibitor. Thus, the screening is more likely to produce a meaningful differential signal.

### Example 1

### Identification of Genes in HeLa Cells that are Involved in FAS Antibody Sensitivity or Resistance

The Achilles Heel Method was applied to HeLa cells treated with anti-Fas antibody in order to identify genes that when knocked-out cause sensitization or resistance of HeLa cells to the action of anti-Fas antibodies.

HeLa cells are derived from a human cervical carcinoma and were used in the original TKO method [Deiss and Kimchi, 1991]. HeLa cells were used as an exemplar of the method of the present system as they are easily grown in culture, are easily transfected and respond to anti-Fas antibody treatment.

Anti-Fas antibody (Kamiya Biomedical Company, Seattle, Wash., catalog number: MC-060) is directed against Fas/CD95/Apo-1, a transmembrane receptor that is known to signal a death response in a variety of cell types. This antibody is an activating antibody, that is, the binding of the antibody mimics the effects of binding of the natural FAS-ligand. Applying the appropriate dose to responding cells has been shown to lead to induction of cell death (Deiss et al., 1996). HeLa cells respond to this treatment.

In this Example, genes are identified that regulate the sensitivity of HeLa cells to killing by anti-Fas antibody. Specifically, one group of genes are identified whose loss sensitizes HeLa cells to anti-Fas treatment. Another group of genes are identified whose loss protects HeLa cells from anti-Fas treatment.

The outline of the procedure is as follows:

1. HeLa cells were transfected with a GIE cDNA episomal vector library.

2. Cells containing GIE expression vectors were isolated by selection with Hygromycin. Since the vector contains the Hygromycin resistance marker, the selection of the transfected cultures with Hygromycin generated a population of cells which contain the GIE expression vectors.

3. Aliquots of this pool of cells were treated with anti-Fas antibody under two different experimental conditions. It should be noted that several different conditions could be screened at the same time.

a. Treatment with a sub-lethal dose of anti-Fas antibody (10 ng/ml). Cells that are super-sensitive to treatment with anti-Fas antibody were killed, the majority of the population which is resistant to the treatment proliferated, and cells that are under-sensitive proliferated at a higher rate.

b. In the second condition, the cells were treated with a lethal dose of anti-Fas antibody (100 ng/ml). The cells were harvested at 24 hours, before the majority of cells had been killed. In this case, applicants were looking for similar events but the different conditions used may highlight better the resistance phenotype.

4. Aliquots of the cells just before the treatment with anti-Fas antibody (unselected) and just after the treatment period with anti-Fas antibody (selected) were harvested. The DNA contained in each cell population was extracted.

5. The GIE cDNA inserts contained in these DNA samples were preferentially amplified through the use of PCR (see details below). For the purpose of cDNA microarray analysis the sequence of the T7 RNA Polymease promoter was added at the 5′ region of one of the primers (see details below).

6. The pools of GIE cDNA fragments that were derived from cells after treatment were subtracted from those before treatment (see details below). This generated a set of cDNA fragments that were present in cells before treatment but were absent after treatment. These fragments are good candidates for sensitizing cDNA fragments. In other words, it is likely that expression of some of these fragments leads to the inactivation of genes which causes cells to become super-sensitive to anti-Fas antibody treatment. These super-sensitive cells are quickly killed at a lower dose of anti-Fas antibody or more rapidly than the majority of cells. These cells are therefore lost from the treated cultures but are present in the untreated population. Likewise, the plasmids inducing this super-sensitivity are present in the cells before treatment but are absent from the cell sample taken after treatment. Thus, these fragments are identified during the subtraction. It should be noted that subtraction in the other direction, namely, subtracting GIE fragments derived from unselected cell from those from selected cells, would identify GIEs enriched after selection. Such a process would allow the detection of cDNA fragments conferring resistance from Fas-induced apoptosis.

7. The cDNA fragments generated by the subtraction were cloned into the original expression vector. Appropriate restriction enzyme sites were generated or maintained during the subtraction procedure so that the recloned construct is exactly identical to the construct in the originally transfected cells. The sequence of the isolated cDNA fragments was determined. From one such fragment the entire sequence of gene 606 was determined.

8. GIEs derived from the original library were cloned into the plasmid pBluescript KS+. A set of 2880 clones from this GIE pBluescript library were used for printing a cDNA microarray. Briefly, the GIE insert of each clone was individually amplified by PCR, the product purified on a PCR purification column, and used for printing. During this process each bacterial clone containing the pBluescript plasmid carrying the GIE was frozen for storage. The location of each clone on the chip is precisely registered to allow smooth correlation between the results of cDNA microarray hybridization and the identity of the clones.

9. The GIE pools recovered from the selected and unselected cells, which contained the T7 RNA polymerase promoter upstream or downstream, were used. The two GIE derived amplified cDNA pools were separately transcribed into RNA by a reaction with T7 RNA polymerase. The RNA was then used for reverse transcription labeling reactions. The RNA derived from the unselected was used for labeling with Cy3 and the RNA derived from the selected cells was used for labeling with Cy5. The resulting labeled cDNA pools were mixed and hybridizaed to the cDNA microarray.

10. The GEMTools program (Incyte Pharmaceuticals, Inc.) was used to quanify the hybridization results and determine which printed clones were differentially represented after the selection. Clones that gave a value of Cy3/Cy5 equal or greater than +2 and clones that gave a value of Cy3/Cy5 equal or lower than −2 were indentified and the matching frozen bacterial clones used to prepare plasmids for sequencing. The inserts of these plasmids were sequences and the identity of the sequences, as either known or novel genes, was determined by bioinformatics analysis programs such as BLAST (NCBI).

11. GIE expression plasmids containing selected cDNA inserts that were identified by the AHM were individually re-transfected into HeLa cells and the transfectant cells were assayed for sensitivity to anti-Fas antibody treatment.

Specific Materials and Methods

a. Transfection

HeLa cells were transfected with GIE cDNA library cloned in the episomal vector, GIE expression vector pTKO-1. This is the same library described in Deiss and Kimchi [1991]. One million cells plated in a 100 mm dish were transfected with DNA containing the GIE cDNA library, by using the Superfect reagent (Qiagen, Santa Clarita, Calif.) as suggested by the manufacturer. Two days following transfection, cells were treated with Hygromycin B (200 g/ml) (Calbiochem-Novabiochem Corporation, La Jolla, Calif.). Following two weeks of selection, the population of cells was completely resistant to Hygromycin B.

b. FAS Antibody Selection

The Hygromycin B selected cells were plated in triplicate at a density of 2.5×10⁶ cells per 150 mm dish in the absence of Hygromycin B. One plate was treated with anti-Fas antibody at 10 ng/ml (clone CHI-11 Kamiya Biomedical Company, Seattle, Wash.) for five days, the second plate was treated with 100 ng/ml of anti-Fas antibody for 24 hours and the third plate was untreated for 24 hours.

c. Recovery of Episomal Vector from Cells

Following the treatments, the cells were harvested by washing twice with ice cold PBS (NaCl 8g/liter; KCl 0.2g/liter; Na₂HPO₄ 1.44 g/liter; KH₂PO₄ 0.24 g/liter; final pH of solution adjusted to pH 7.4 with HCl) and concentrated by centrifugation (15,000× g for 15 seconds). DNA was extracted by using solutions P1, P2 and P3 from the Qiagen Plasmid Purification Kit (Qiagen, Santa Clarita, Calif.). The cell pellet was resuspended in solution P1 (50 mM Tris-HCl, pH 8.0; 10 mM EDTA; 100 ig/ml RNase A) then mixed with solution P2 (200 mM NaOH, 1% SDS) and incubated five minutes at room temperature. Solution P3 (3.0M Potassium Acetate, pH 5.0) was added and incubated two minutes at room temperature, followed by a ten minute centrifugation at 15,000× g. The clear supernatant was mixed with an equal volume of isopropanol and centrifuged at 15,000× g for ten minutes. The precipitated DNA was resuspended in water and stored frozen until use.

d. Amplification of cDNA Inserts (GIEs)

For PCR amplification of the cDNA inserts contained in these DNA preparations, the following reaction was set in a total volume of 100 μl: 1 μl of the DNA, 200 μlM of dATP, dGTP, dCTP, dTTP, 500 ng each of primers prLPD#64 (SE(SEQ ID No:2) and prLPD#65 (SEQ ID No:3); 10 mM Tris-HCl pH 9.0; 0.1% Triton x-100; 1.0 mM MgCl and 1 unit of Taq DNA polymerase (Gibco/BRL, Gaithersburg, Md.). This reaction was incubated in a Thermocycler 2400 (Perkin-Elmer, Foster City, Calif.) according to the following protocol: First, the reaction was heated to 94° C. for five minutes, then was cycled 25 times using the following three temperatures: 58° C. for one minute, 72° C. for five minutes, 94° C. for one minute. After 25 cycles, the reaction was incubated at 72° C. for seven minutes. This resulted in amplification of the cDNA inserts. The prLPD#64 and prLPD#65 primers were designed such that the end of the cDNA insert that is proximal to the promoter in the pTKO-1 vector is exactly flanked by a HindIII restriction site (this site is present in the vector) and the end of the cDNA that is distal to the promoter in pTKO-1 vector contains a BamHI restriction site. The BamHI site was created by altering a single base in the sequence immediately adjacent to the distal cDNA insert site (prLPD#65), by PCR. When the library was generated [Deiss and Kimchi, 1991], this site distal to the promoter was generated by the fusion of a BamHI restriction site (derived from the cDNA fragments) and a Bgl II site (derived from the vector). This fused site is resistant to cleavage by either enzyme, but a single base change restored the cleavage by BamHI. Thus, the amplified cDNA fragments are flanked by a HindIII restriction site on the promoter proximal side of the cDNA and by a BamHI site on the promoter distal side. This allowed the exact re-cloning of the fragments into the pTKO-1 expression vector with exact conservation of sequence and orientation.

e. Subtraction

Following the PCR reaction, the mixture was cleaved with BamHI and HindIII (Gibco/BRL, Gaithersburg, Md.) as described by the manufacturer. The digestion products were purified using the Wizard PCR Prep Kit (Promega, Madison, Wis.). This generated cDNA inserts with HindIII and BamHI ends. These nucleic acid fragments were subjected to subtraction using the PCR-Select Kit (Clontech, Palo Alto, Calif.) according to the instructions of the manufacturer with the following modifications. The driver was the PCR products derived from the untreated samples and two testers were used. The first tester was derived from cells treated with 10 ng/ml anti-Fas antibody and the second tester was derived from cells treated with 100 ng/ml of anti-Fas antibody. First modification: the subtraction is done between dsDNA pools so no cDNA synthesis is required. The fragments generated from the previous step were used directly in the subtraction. Thus, applicants began at Step IV F3 in the instructions (preparation of the adapter ligated tester cDNA). The second modification was the replacement of the blunt end ligation of adapter 1 and adapter 2R with cohesive end adapters. These cohesive end adapters were ligated to the BamHI and HindIII cleaved PCR fragments generated in the step above. The cohesive ligation is usually more efficient than blunt end ligation and since applicants use cDNA flanked by different restriction sites allowing the orientation of the fragments to be maintained when recloning the subtracted products. If blunt end ligation is used, it does not allow distinguishing one end from the other and applicants would not be able to determine the relative orientation of the cDNA in the original expression cassette.

The manual supplied by the manufacturer with the kit was followed from the point of ligation of the adapters to the tester (Section IV F3 in the Manual). The tester was taken for adapter ligation. The initial hybridization included 0.9 μg of the driver and 0.03μg of the adapted ligated tester. At the conclusion of the subtraction, a final PCR reaction wes performed using nested PCR primer 1 (prLPD#86) and nested PCR primer 2R (prLPD#87). This material contained the cDNA fragments that were present in the untreated sample but absent from the treated samples. The products of this PCR reaction were re-cloned into the anti-sense expression vector.

f. cDNA Microarray Analysis

For analysis on cDNA microarray the PCR amplification described in “d” was performed with the following modifications. Two consecutive PCR amplifications were performed. First reaction: a 50μl reaction includes 1 μl of lysate DNA, 200 μM of dATP, dGTP, dCTP, dTTP, 3 units Expand High-Fidelity, 1× (final concentration) reaction buffer (as supplied by Roche with the enzyme) and 20 pmol/μl each of primers prLPD#207 and prLPD#78. Reaction was carried out at 94° c. for 2 minutes followed by 10 cycles of 94° c. for 30 seconds, 60° c. for 30 seconds, and 68° c. for 5 minutes. The reaction was terminated by incubation at 68° c. for 5 minutes. PCR products were purified on a PCR purification column. Second reaction: 10 μ1 from the purified products of the first reaction, 200 μM of dATP, dGTP, dCTP, dTTP, 3 units Expand High-Fidelity, 1× (final concentration) reaction buffer (as supplied by Roche with the enzyme) and 20 pmol/μl each of primers prLPD#205 and prLPD#78. Total reaction volume is 50 μl. Reaction was carried out at 94° c. for 2 minutes followed by 20 cycles of 94° c. for 30 seconds, 60° c. for 30 seconds, and 68° c. for 5 minutes. The reaction was terminated by incubation at 68° c. for 5 minutes. PCR products were purified on a PCR purification column. These PCR reactions produced cDNA inserts flanked by the T7 RNA polymerase promoter sequence on the side proximal to the SV40 polyadenylation signal on the episomal vector.

Another option is to produce cDNA inserts flanked by the T7 RNA polymerase promoter sequence on the side proximal to the promoter region on the episomal vector. This is done by performing the first PCR with different primers prLPD#105 and prLPD#206 and the second PCR with primers prLPD#105 and prLPD#205. The reaction conditions are exactly the same.

The purified PCR products are used as templates for producing RNA by a T& RNA polymerase reaction as follows: A 50 μl reaction contained 15 μl PCR is products, 0.4 mM each of rATP, rCTP, rGTP, rUTP, 50 units of T7 RNA polymerase (New England Biolabs), 1× buffer (supplied with the enzyme, and 40 units RNAsin (promega). Reaction was incubated at 37° c. for 4 hours. The reaction was then cleaned by phenol/chloroform extraction and precipitation with ethanol. For labeling with Cy3 or Cy5 the GEM Bright kit (Incyte Pharmaceuticals, Inc) was used. An amount of 0.6 μg of RNA was used and the protocol supplied with the kit was followed. After labeling the products were purified on a PCR purification column. Control (Cy3 labeled) and treated samples (Cy5 labeled) were mixed and hybridized to the cDNA microarray. All of the methods of relating to cDNA microarray analysis are well known in the art and were followed.

g. Cloning of Subtraction Output cDNA Fragments

Re-cloning of the subtracted fragments was accomplished by cleaving the subtracted population with BamHI and HindIII and purifying the cleaved products with the Wizard PCR Prep Kit (Promega Madison, Wis.). The cleaved products were then directly cloned into the pTKO1-DHFR vector between the HindIII and BgIII sites. In some cases the products were cloned into the pBluescript KS+vector cleaved with BamHI and HindIII. This replaced the DHFR sequences with the cDNA. This is precisely the procedure that was used to generate the GIE cDNA expression library. Thus, the fragments that were generated by the subtraction were exactly re-cloned into the original GIE expression vector that was used to transfect cells at the beginning of the procedure. The re-cloned constructs exactly duplicate the constructs that were present in the library. The re-cloned constructs were introduced into bacteria and DNA was extracted from the bacteria using conventional methods.

h. Sequencing and Annotation of the cDNA Inserts

These DNA preparations were used as a template for sequencing in order to determine the nucleotide sequence of the isolated cDNA inserts. Primer prLPD#51 for pTKO-1 and primer M13 for pBluescript were used in Automated sequencing using Applied Biosystems 377XL. DNA sequencer with PerkinElmer Dye Terminated Sequencing Kits (Perkin-Elmer, Applied Biosystems Division, Foster City, Calif.). The clones were sequenced using primer prLPD#51 which anneals close to the edge of the cDNA which is distal to the promoter in the antisense expression cassette. Thus, in the case that the sequence matches the sense strand of a known gene then the insert is in the antisense orientation. Sequences were compared to the combined nonredundant database and the dbest compiled at the NCBI using the Blastn program with default parameters (Internet address: http://www.ncbi.nim.nih.gov/cgi-bin/BLAST/nph-blast?Jform=0). The sequences of the polynucleotides determined by the above method are listed in Table D.

i. Confirmation of the Biological Activity of Individual cDNA Inserts

pTKO-1 plasmids carrying the re-cloned inserts were transfected into HeLa cells to confirm their ability to induced super-sensitization (or resistance) to antiFas antibody treatment in HeLa cells. HeLa cells were transfected with 15 μg of plasmids or control vectors as described for transfection of the original library. The cells were selected for two weeks for resistance to Hygromycin B treatment (200 μg/ml). This selects for cells which contain expression vectors. One million cells were plated in a 100 mm dish and treated with anti-Fas antibody. Effects of anti-Fas antibody on the transfected cultures were quantified by MTT assays as described by the manufacturer (Sigma, St. Louis, Mo.). The magnitude of the effect of the cDNAs was determined by comparison to the effects observed with the control pTKO-1 vector.

### Example 2

### Identification of Genes Invoved in the FAS Pathway and Inhibitors of the Genes

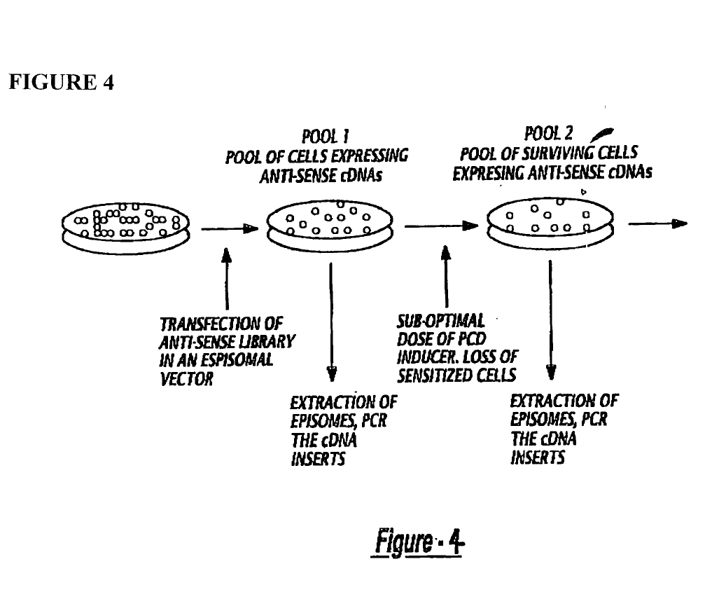

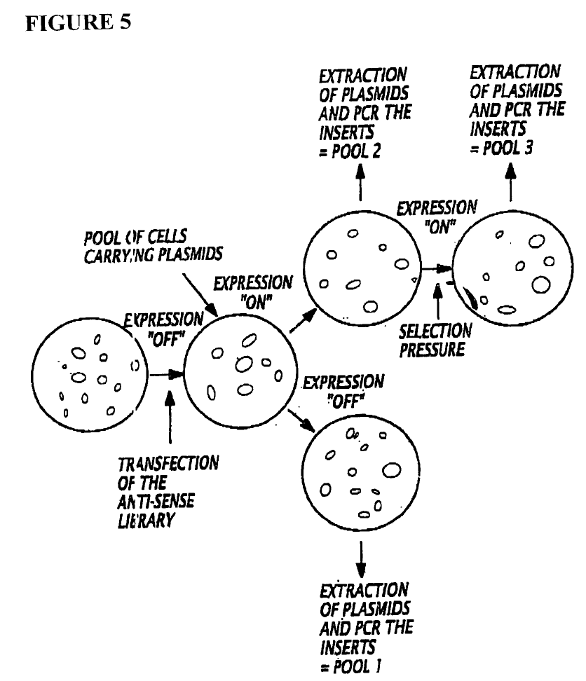

The Achilles Heel Method utilizes functional profiling as diagrammed in FIG. 4. The first step consists of introducing an GIE expression library (Deiss and Kimchi, 1991) into target cells to generate a pool of cells, each expressing a different GIE fragment (Pool 1). Then, the transfectants are treated with a sub-optimal dose of a PCD inducer and the surviving cells are collected (Pool 2). AHM can identify both sensitizing and protecting GIE cDNAs, in this example sensitizing events were targeted. Cells containing inactivation events that sensitize them to killing are preferentially lost from Pool 2. Consequently, the GIE cDNAs contained in the sensitized cells are depleted from Pool 2. The “sensitizing” cDNA inserts that are present in Pool 1 but depleted from Pool 2 are identified by two methods, subtraction or hybridization to cDNA microarray. Following the subtraction of Pool 2 cDNAs from Pool 1 cDNAs, the potentially sensitizing cDNAs are cloned in the episomal expression vector exactly the same original orientation in which they were in the episomal vector in the cell library used for selection. The GIE cDNA containing episomes are individually transfected into target cells in order to confirm their ability to render the cells more sensitive to the killing inducer. Alternatively, Pool 1 and Pool 2 cDNAs are labeled and used as probes for hybridization to cDNA microarrays. Computer analysis identifies the cDNAs depleted from Pool 2. As for subtraction, following cDNA microarray analysis cDNA clones showing depletion from pool 2 are cloned in the episomal expression vector exactly the same original orientation in which they were in the episomal vector in the cell library used for selection. It should be noted that the clones printed on the cDNA microarray were derived from the GIE library used in the selection process. In both cases, subtraction and cDNA microarray analysis, “function profiling” is being employed to identify signal pathway inhibitors. Recently, similar “function profiling” methods have been described for genetic analysis of S. cerevisae (Shoemaker et al. 1996; Smith et al. 1996) These methods are well suited to yeast since they require prior knowledge of gene sequence and the ability to generate haploid cells. By contrast, AHM does not require a priori knowledge of any gene sequence or haploid cells. Thus, AHM is a powerful genetic tool for “function profiling” in mammalian cells. Moreover, AHM can be easily scaled up to generate “function profiles” of all expressed human genes.

AHM was used to identify inhibitors of the Fas induced programmed cell death (PCD) pathway. Fas is a trans-membrane death receptor of the TNF super family. The binding of Fas ligand to Fas results in the cascade of events that lead in most cell types to apoptosis. Fas induced killing is utilized in different physiological processes as follows: (for review see (Nagata 1997)): elimination of auto-reactive T-cells, tumor induced immune suppression and destruction of virally infected cells, transformed cells and b-cells in cases of Insulin Dependent Diabetes Melitus (IDDM). In addition, activation of the Fas pathway has been suggested to play a role in liver damage, brain damage, arteriosclerosis and tumor suppression. Modulation of the Fas pathway has clinical implications in animal models: inhibition of Fas induced PCD by caspase inhibitors limits liver damage in mice and acceleration of Fas induced killing ameliorates the auto-immune phenotype of gld mice. Thus, identifying regulators of the Fas pathway that can be used as targets for drug development can have great clinical impact.

For the identification of inhibitors of Fas induced cell death, AHM was applied to HeLa cells that were treated with sub-lethal dose of Fas agonistic antibody. The later mimics the binding of Fas ligand to Fas and induces apoptosis. “Function profiling” (also termed functional profiling) was performed to identify “sensitizing” cDNA fragments by using subtraction and cDNA microarray analysis. cDNA inserts from Pool 2 were subtracted from Pool 1 cDNAs and the recovered cDNAs were further analyzed. Many of the cDNAs that were individually transfected into HeLa cells conferred increased sensitivity to Fas induced killing cells, ranging between 2.9 to 5.3 fold. These fragments include many novel sequences and three fragments of previously described genes. One of the cDNA inserts is an anti-sense fragment of human Basic Fibroblast Growth Factor (FGF-2, bFGF) and the other is an anti-sense fragment of the cap-n-collar b-zip transcription factor NF-E2 related factor 2 (NRF2).

bFGF is a potent survival factor that plays a role in development, angiogenesis, and in cell migration. Previous reports have shown that down regulation of bFGF by anti-sense expression or by blocking antibodies result in loss of a transformed phenotype, reduced tumor growth and reduced angiogenesis. Five different polypeptides of 34 kD, 24 kD, 22.5 kD, 22 kD and 18 kD are translated from the human bFGF gene, initiating at different sites and terminating at the same position. The anti-sense cDNA fragment isolated in the subtraction is 295 nucleotides long and corresponds to nucleotide 873 to 1167 of the bFGF gene (Genebank Accession Number NM_{—}002006). It spans the last 60 nucleotides of the coding region (shared by all bFGF polypeptides) and a portion of the 3′ un-translated region.

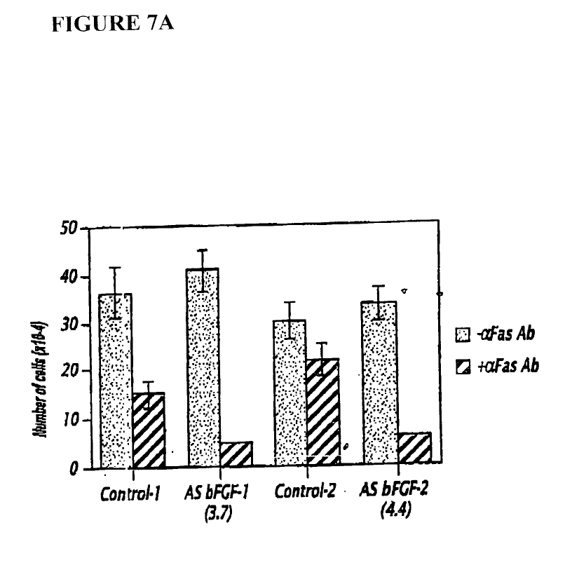

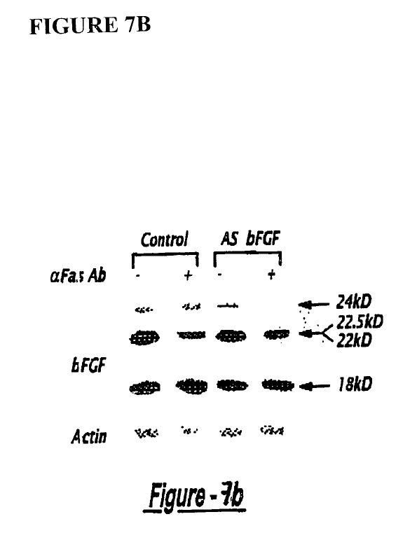

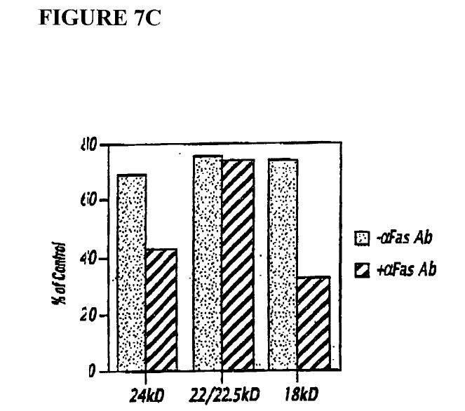

In order to confirm that anti-sense bFGF confers sensitivity to Fas, pools of cells transfected with control vector (harboring no insert) or with anti-sense bFGF were generated and treated with a sub-optimal dose of anti-Fas antibody. Analysis of two independent pools of transfectants demonstrates that under conditions that result in 59% and 29% killing of the vector transfected cells, anti-sense bFGF transfected cells are 3.7 and 4.4 fold more sensitive to killing (FIG. 7A,). This significant increase in sensitivity of antisense bFGF transfected cells was reproducible in six independent pools of transfectants. It is not due to altered growth rate of the anti-sense bFGF transfected cells (compare the number of untreated cells in the control vector transfected pools to the number of untreated cells in the anti-sense bFGF transfected pools) or to a non-specific increase in sensitivity of anti-sense transfected cells to Fas induced killing since anti-sense cDNAs were previously isolated that render transfected cells resistant to Fas induced apoptosis. Complementary to the bioassay experiments, quantitative Southern analysis of Pool 1 and Pool 2 indicates that the abundance of anti-sense bFGF cDNA is reduced by 1.9 fold in Pool 2 of cells surviving sub-lethal dose of anti-Fas antibody, compared to Pool 1.

Western blot analysis of control vector transfected cells as well as anti-sense bFGF transfected cells revealed four polypeptides of 24 kD, 22/22.5 kD and 18 kD, while the 34 kD form is not detected by the antibody used (FIG. 7B). Quantitative analysis of the relative level of bFGF forms revealed that in the absence of anti-Fas antibody, expression of anti-sense bFGF results in reduction of approximately 25%-30% in the levels of each of the detected forms (FIG. 7C). Interestingly, in cells treated with anti-Fas antibody a more significant reduction in the levels the 24 kD form and 18 kD is observed, 57% and 66% respectively, while the reduction of the levels of the 22/22.5 kD is not altered. Selective reduction in the level of some of the bFGF forms by an antisense fragment that overlaps the coding region of all the bFGF polypeptides can be due to a network of feedback regulation loops as previously reported for some bFGF forms.

While previous studies has shown that over-expression of the 34 kD form protects cells from serum deprivation induced killing and over-expression of the 24 kD form protects cells from ionizing radiation, here it is demonstrated that bFGF is an inhibitor of Fas induced apoptosis, as identified by AHM.

The second inhibitor of the Fas pathway that was identified by AHM is the cap-n-collar b-zip transcription factor NF-E2 related factor 2 (Nrf2). Nrf2 activates the transcription of phase II detoxifying enzymes such as NAD(P)H quinone oxireductase (NQO1) and Glutathione S-transferase (GST) by direct binding to the Antioxidant Response Element (ARE) in the promoter of these genes. Studies of Nrf2 null mice indicate that Nrf2 is essential for the transcriptional activation of phase II enzymes in response to an anti-oxidant. NQO1 and GST act in concert with phase I detoxifying enzymes (such as cytochrome p-450 monooxygenase) to mediate the cellular detoxification of xenobiotics. In the absence of Nrf2, this coordinated detoxification is impaired and toxic products from phase I reactions can accumulate. In the AHM screen an anti-sense fragment of Nrf2 corresponding to nucleotide 145 to 972 of the human Nrf2 (GeneBank Accession Number S74017, (Moi et al. 1994)) was recovered.

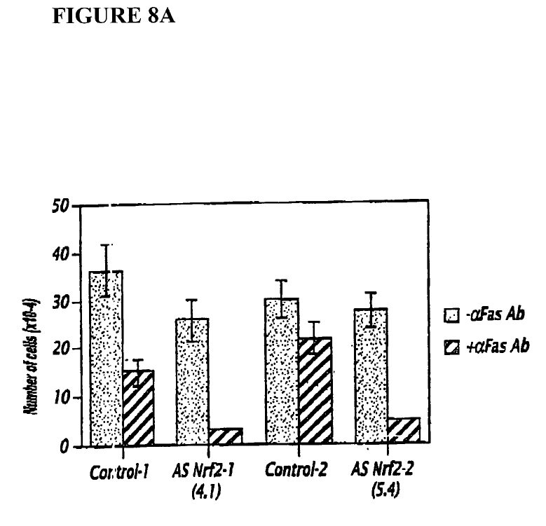

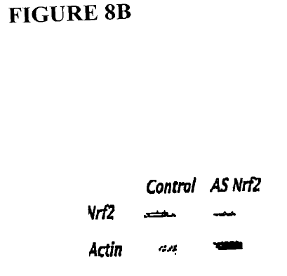

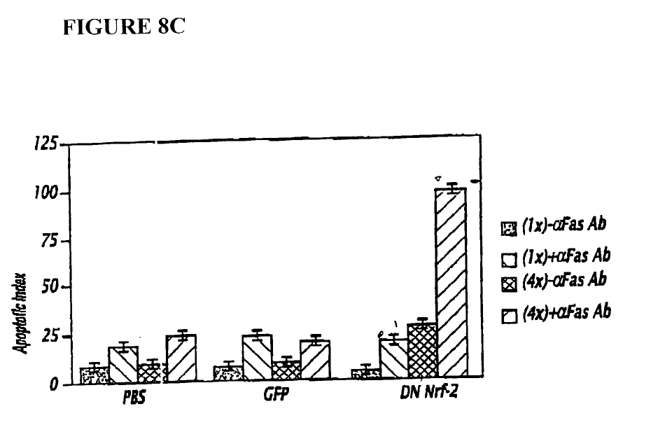

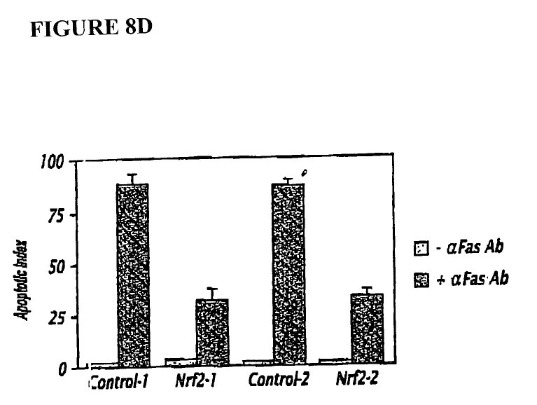

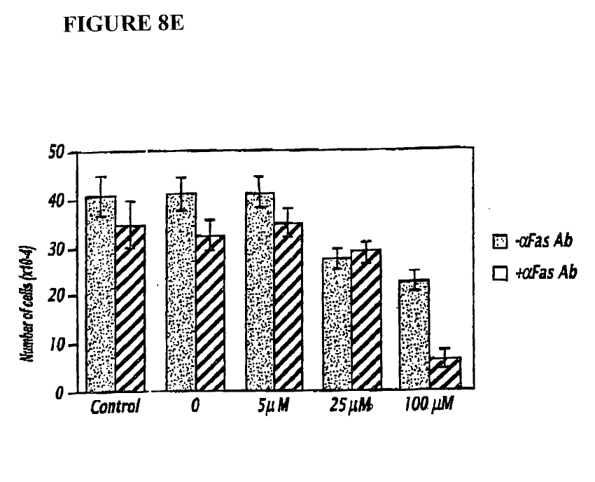

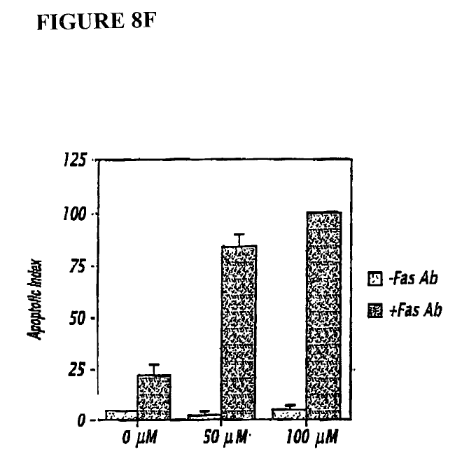

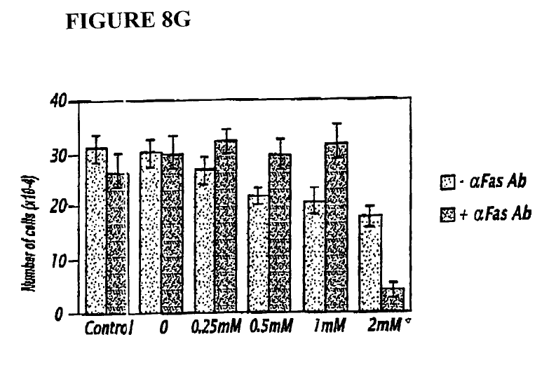

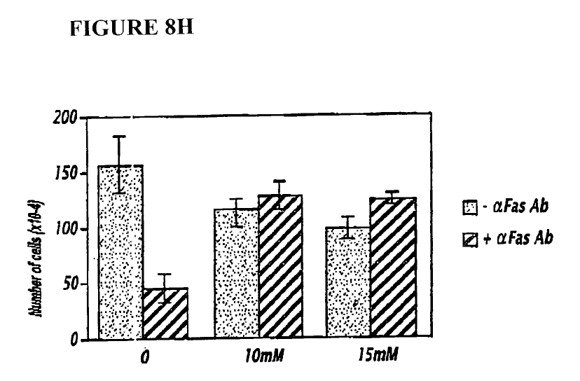

Bioassays of two pools of HeLa cells transfected with anti-sense Nrf2 clearly demonstrates that anti-sense Nrf2 render the cells 4.1 and 5.4 fold more sensitive to Fas induced apoptosis (FIG. 8A). Again, this increased sensitivity is not a result of impaired growth, since there is only limited alteration in the growth rate of anti-sense Nrf2 transfected cells (FIG. 8A). Sensitization by anti-sense Nrf2 was reproducible in seven independent pools of transfectants. Western blot analysis indicated a significant 3.8 fold reduction in the level of Nrf2 protein in the anti-sense Nrf2 transfected cells (FIG. 8B).

An alternative method of down regulation of Nrf2 was employed to confirm that inhibition of Nrf2 can sensitize cells to Fas induced killing. Nrf2 is a transcription factor that contains an amino terminal trans-activation domain and a carboxyl terminal DNA binding domain. A dominant negative (DN) version of Nrf2 consisting of the DNA binding domain but lacking the trans-activation domain was generated. Ohtsubo et al. had shown that such a construct effectively inhibits the ability of wild-type Nrf2 to activate transcription (Ohtsubo et al., 1999). In order to efficiently introduce this dominant negative Nrf2 fragment into cells, a membrane solubilization domain of HIV TAT was added to it. The membrane soluble DN Nrf2 was produced in bacteria and used to treat HeLa cells (FIG. 8C). In the presence of low dose of vehicle (1×) or high dose vehicle (4×), an apoptotic index of approximately 10% is observed in the untreated cultures and 25% in cultures treated with anti-Fas antibody. Similarly, the administration of low or high dose of the control membrane soluble Green Fluorescent Protein (GFP), did not alter the response of cells to treatment with anti-Fas antibody. The addition of low dose of DN Nrf2 (1×) did not alter the response to anti-Fas antibody. However higher dose of DN Nrf2 (4×), significantly sensitizes cells to killing induced by anti-Fas antibody while only modestly affected cell viability. Thus, using a different method, totally independent of anti-sense expression, it was confirmed that inhibition of Nrf2 leads to sensitization of cells to Fas mediated apoptosis. Moreover, the membrane soluble version of DN Nrf2 can be used as a drug to sensitize human cells to apoptosis.

Further testing to determine whether over-expression of Nrf2 can protect cells from Fas induced apoptosis was performed. The coding region of Nrf2 was cloned into a retroviral vector. Cells infected with the retrovirus were selected for resistance to puromycin, since the retrovirus carries a puromycin resistance marker. Two pools of puromycin resistant cells, as well as the corresponding control vector infected cells, were assayed for their response to Fas induced apoptosis. The results shown in FIG. 8D demonstrate that expression of Nrf2 protects cells from Fas induced apoptosis. Untreated cells show a very low apoptotic index. Anti-Fas antibody treatment of cells infected with vector alone (Control-1, -2) results in apoptotic index of 89% and 87%, while cells infected with an Nrf2 encoding retrovirus (Nrf2-1, -2) show apoptotic index of only 32% and 34% respectively.

The role of Nrf2 as an inhibitor of the Fas pathway was further validated by pharmacological agents. It was predicted that treatment of cells with Dicumarol, an inhibitor of GST and NQO1 can sensitize cells to Fas induced apoptosis, since Nrf2 up-regulates the levels of GST and NQOL. As shown in FIG. 8E, HeLa cells treated with 100 mM Dicumarol are 2.8 fold more sensitive to Fas induced killing compared to cells treated with vehicle as measured by the number of viable cells. This result was further validated by staining the cells with DAPI, which is commonly used to detect apoptotic cells, as measured by chromosomal condensation and fragmentations, hallmarks of apoptosis (FIG. 8F). Apoptotic index of approximately 25% is observed in cells treated with anti-Fas antibody in the presence of vehicle (0 uM) and approximately 100% killing is observed in cells treated with anti-Fas antibody and 100 uM Dicumarol Thus, Dicumarol significantly sensitizes HeLa cells to Fas induced programmed cell death.

Since Nrf2 regulates genes involved in phase 11 detoxification, testing was conducted to determine whether other activities involved in detoxification can also influence the sensitivity of cells to Fas induced apoptosis. It has been reported that the detoxification of some compounds involves the phase 11 gene GST and the action of a sulfinpyrazone sensitive export pump (Morrow et al. 2000). Then it was directly tested whether sulfinpyrazone treatment of cells can sensitize to Fas induced apoptosis. As is shown in FIG. 8G, treatment of cells with 2 mM sulfinpyrazone strongly sensitizes cells to the effect of Fas induced killing compared to treatment with vehicle alone, by approximately 4 fold. Thus, treatment of cells with sulfinpyrazone can have clinical benefits in situations where enhanced cell killing can be beneficial.

Since down regulation of Nrf2 sensitizes cell to Fas induced apoptosis, it was questioned whether increasing any of the activities induced by Nrf2 protects HeLa cells from apoptosis. Nrf2 up-regulates GST that conjugates glutathione to the reactive products of phase I detoxification. It was predicted that increased activity of GST protects cells from Fas induced apoptosis. GST activity was elevated by treating HeLa cells with the glutathione precursor N-acetyl Cysteine (NAC) that increases the glutathione pool. As shown in FIG. 8H, NAC strongly protects HeLa cells from Fas induced apoptosis as previously reported for microglia, neutrophils and T-cells (Delneste et al. 1996; Watson et al. 1997; Spanaus et al. 1998).

Thus, by using AHM, Nrf2 was identified as an inhibitor of Fas induced PCD in HeLa cells and this result was validated by genetic and pharmacological approaches. Down regulation of Nrf2 sensitizes to killing while over-expression of Nrf2 protects from Fas induced apoptosis.

A technically simpler alternative to “function profiling” by subtraction is analysis by filter microarray. The relative abundance of cDNAs was measured in Pool 1 and in Pool 2 by radio-labeling each pool and hybridizing each of the probes to a cDNA microarray filter containing approximately 4,000 different known human genes. Dark green spots indicate cDNAs absent in Pool 2, representing “sensitizing” anti-sense cDNAs. The corresponding genes are predicted to be survival factors that inhibit Fas induced apoptosis. Dark red spots indicate cDNAs that are enriched in Pool 2. These genes are positive mediators of killing and their inactivation by anti-sense results in resistance to PCD. The abundance of such anti-sense cDNAs is therefore increased in Pool 2 that is comprised of cells that survived Fas induced apoptosis. Most of the spots are of intermediate color indicating only modest or no changes in abundance in Pool 2 relative to Pool 1. The abundance of the majority of cDNAs is not changed. However, a small number of cDNAs are depleted from Pool 1 by 2 folds or more.

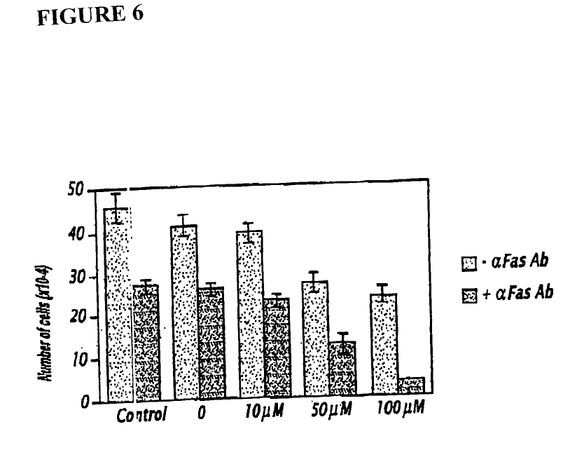

Also validated was the role of casein kinase 1 alpha (CSNK1A1) CSNK1A1 as an inhibitor of the Fas pathway. Since CSNK1A1 is substantially reduced in Pool 2 by 5.2 fold, down regulation of CSNK1A1 by its pharmacological inhibitor CKI-7 (Chiajiwa et al. 1989) sensitizes cells to FAS induced apoptosis. As shown in FIG. 6, HeLa cells treated with CKI-7 are 3.9 fold more sensitive to FAS induced PCD than the untreated cells. Again, it was validated, by a chemical inhibitor, that a gene identified by the AHM method involving the cDNA microarray analysis is an inhibitor of the Fas pathway

Materials and Methods:

Anti-Sense Transfection and Bioassays:

HeLa cells (2×10⁶ cells/100 mm plate) were plated 20 hours prior to transfection with either 17 ug of either GIE expressing vector or control vector harboring no cDNA insert, by calcium phosphate. Forty eight hours post transfection cells were treated with 200 ug/ml Hygromycin B (Calbiochem-Novabiochem) for two weeks. For bioassays, GIE-vector transfected cells or control vector transfected cells (1.6 ×10⁵ cells/well in 6 wells plates) were plated 20-24 hours prior to the treatment with 200 ng/ml anti-Fas antibody (clone CH-11, Kamiya Biomedical Company). The number of viable, trypan blue (Gibco/BRL) excluding cells that remained attached to the plate following rinsing with PBS was counted

24 hours post treatment. Nrf2 Infection and Bioassays:

To generate HeLa cells that over-express Nrf2, the human Nrf2 cDNA (Moi et al. 1994) was cloned into the retrovirus expression vector pBABEpuro (Morgenstern and Land 1990) by standard methods. The plasmid was transiently transfected into X and virus was collected at 36, 48, 60 and 72 hours post transfection. At each time point, freshly collected virus stock was filtered through a 0.45 filter, mixed with 4 gr/ml polybrene (Sigma/Aldrich) and applied to HeLa cells. 24 hours after the last addition of the virus, the cells were subjected to selection in 1 gr/ml Puromycin (Sigma/Aldrich). The pool of Puromycin resistant colonies was expanded. For bioassays (1.6×10⁵ cells/well in 6 wells plates) were plated 20-24 hours prior to the treatment with 200 ng/ml anti-Fas antibody (clone CH-11, Kamiya Biomedical Company). Three days later the cells were fixed by slowly adding ⅓ volume of formaldehyde (37%) to the medium. Twenty four hours later the cells were gently washed twice with dH₂O and stained with 2.5 g/ml DAPI (4′,6-Diamidino-2-phenylindole, Sigma/Aldrich) in PBS for 5 minutes followed by two washes in dH₂O. Stained cells were viewed by Leica DMIRB fluorescent inverted microscope. Apoptotic index was calculated as the ratio of number of dead cells (containing condensed and/or fragmented chromosomes) to the total number of cells. Average apoptotic index calculated from ten fields from duplicate wells is presented.

Generation of Membrane Permeable Dominant-Negative Nrf2 and Bioassays:

Human Nrf-2 protein(amino acid 367 to 589) was cloned between the KpnI and EcoRI sites of a bacterial expression vector pTAT-HA vector. The GFP control protein expressed from the PTAT-HA-GFP vector as well as the Nrf2 dominant negative recombinant polypeptide were produced in bacteria as described (Schwarze et al. 1999). Briefly, bacteria strain BL-21 (DE-3) pLysS was transformed by the plasmid of interest and an overnight culture was grown at 37° C. Approximately 18 hours later, the culture was diluted into 500 ml of fresh LB media and grown to OD of 0.8-0.9 at 37° C. IPTG was added (0.5 mM final concentration) followed by incubation at 30° C. for additional three hours. The bacterial pellet was washed in 20 ml of PBS. The final pellet was resuspended in 10 ml of buffer Z (8M Urea, 100 mM NaCl, 20 mM HEPES pH 8.0) and sonicated on ice 4×30 sec pulses. The sonicated lysate was centrifuged at 12,000 rpm for 30 minutes at 4° C. Imidazole (20 mM final concentration) was added to the supernatant. The supernatant was then added to a Ni-NTA column (3 ml) in Buffer Z containing 20 mM imidazole. The column had been pre-equilibrated with buffer Z containing 20 mM imidazole. Following binding of the lysate to the column, the column was washed with 50 ml of buffer Z containing 20 mM imidazole. The fusion protein was eluted by washing the column with increasing concentrations of imidazole (100mM, 250 mM, 500 mM and 1 M imidazole) in buffer Z. The protein fraction eluted in 1M imidazole was dialyzed against sterile PBS for 12-16 hours with two changes. Glycerol (10%. final concentration) was added to the dialyzed protein. The concentration of the purified protein was estimated by SDS PAGE compared to standard BSA.

For bioassays, HeLa cells, at 8.3×10⁴ cells/well were plated in 12 wells plates. 20 hours later, cells were treated with various concentrations of recombinant protein or PBS 60 minutes prior to the addition of 200 ng/ml anti-Fas antibody (clone CH-11, Kamiya Biomedical Company) (where indicated). 20 hours later the apoptotic index of treated cells was determined as described for the Nrf2 bioassays.

Western Analysis:

Anti-sense transfected cells or control vector transfected cells (2.5×10⁶ cells/150 mm plate) were plated 24 hours prior to treatment with 200 ng/ml anti-Fas antibody (clone CH-11, Kamiya Biomedical Company). 24 hours post treatment cells were washed with PBS and lysed in RIPA buffer (1% Nonidet P-40, 0.5% sodium deoxycholate, 0.1% SDS, 1 mM PMSF, 2 mg/ml aprotonin and 2 mg/ml pepstatin in PBS). Samples containing 50 g protein were separated by SDS-PAGE and transferred to nitrocellulose membranes. The immunoblots were probed with either anti-Nrf2 antibody (1:100, Santa Cruz, sc722) or anti-bFGF-2 antibody, (1:200, Santa Cruz, sc 079), incubated with goat anti rabbit conjugated to horseradish peroxidase (Pierce) followed by incubation with SuperSignal substrate (Pierce). Following autoradiography, the probes were stripped (Amersham, ECL Western blotting protocols) and the membranes were hybridized with anti-actin antibody, (1:100, Sigma A4700 or A2066). The intensities of the bands were quantified by the National Institute of Health Image program.

Treatment with N-acetyl Cysteine:

HeLa cells (8.3×10⁴ cells/well in 6 wells plates) were plated 20-24 hours prior to treatment with various concentrations of NAC (Sigma/Aldrich) in the presence or absence of 50 ng/ml anti-Fas antibody (clone CH-11, Kamiya Biomedical Company). The number of viable, trypan blue (Gibco/BRL) excluding cells that remained attached to the plate following rinsing with PBS was counted 5 days post treatment.

Treatment with Dicumarol:

HeLa cells, 1.6×10⁵ cells/well were plated in 6 wells plates. 20-24 hours later cells were treated with various concentrations of Dicumarol (Sigma/Aldrich) in 0.2 mM NaOH for 15 minutes prior to the addition of 200 ng/ml anti-Fas antibody (clone CH-11, Kamiya Biomedical Company). The number of viable, trypan blue (Gibco/BRL) excluding cells that remained attached to the plate following rinsing with PBS was counted 17 hours post treatment. Alternatively, the apoptotic index of treated cells was determined as described for the Nrf2 bioassays.

Treatment with Sulfinpyrazone:

HeLa cells, 1.6×10⁵ cells/well were plated in 6 wells plates. 20-24 hours later cells were treated with various concentrations of sulfinpyrazone (Sigma/Aldrich) in 1% DMSO for 15 minutes prior to the addition of 200 ng/ml anti-Fas antibody (clone CH-11, Kamiya Biomedical Company). The number of viable, trypan blue (Gibco/BRL) excluding cells that remained attached to the plate following rinsing with PBS was counted 17 hours post treatment.

Treatment with CKI-7:

HeLa cells, 1.6×10⁵ cells/well were plated in 6 wells plates in duplicates. 20-24 hours later cells were treated with various concentrations of CKI-7 in 1% DMSO (Seikagaku Corporation, Tokyo, Japan) an hour prior to the addition of 200 ng/ml anti-FAS antibody (clone CH-11, catalog number MC-060, Kamiya Biomedical Company). The number of viable, trypan blue (Gibco/BRL) excluding cells that remained attached to the plate following rinsing with PBS was counted 17 hours post treatment.

cDNA Microarray Analysis:

Approximately 500 ng of the PCR products of Pool 1 and Pool 2 (same preparations that were used for the subtraction, before their cleavage by BamHI and HindIII) were labeled with 100 mCi of [³³P] dCTP (3000 Ci/mmole, ICN) by the random primers DNA labeling system (Gibco/BRL), purified (Amersham/Pharmacia, ProbeQuant G50 micro columns) and individually hybridized to Human GeneFilters (GF211, Research Genetics). The filter was pre-hybridized for 40-60 minutes at 68° C. in ExpressHyb Hybridization solution (Clontech), followed by hybridization for 3-5 hours at 68° C. The filter was washed in 2× SSC, 0.05% SDS at room temperature 3-5 times for 10-15 minutes each time followed by 2 washes for 15 minutes each in 0.1× SSC, 0.1% SDS at 55° C. The image was generated by Molecular Dynamics phospho-imager. In between hybridizations, the probe was stripped off by adding boiling solution of 0.5% SDS and incubating at room temperature for 1 hour. Successful removal of probe was confirmed by phosphor-imager analysis. Images processing and calculation of the ratio of the signals of Pool 2 probe to Pool 1 probe were performed by Pathways 11 software (Research Genetics). All the spots that showed significant differential abundance were visually inspected.

### Example 3

Identification and Cloning Process of the Novel Gene Sequence

A gene fragment LPD 606 was identified by the methods described in Examples 1 and 2. From this, the full coding sequence termed gene 606 has been identified by a long and complicated process which is described below.

The initial sequence of the LPD 606 fragment (herein termed the “606 fragment”) was discovered in a functional screen performed using the AHM method as described in PCT publication WO 98/21366. The 606 fragment is a 183 bp fragment (SEQ ID No:18 disclosed in the above PCT publication) and its antisense polynucleotide was initially discovered in the screen.

Using the initial sequence of the 606 fragment, an attempt was made to isolate a full length cDNA by screening of a phage cDNA library (Hela cell line cDNA lib, Clontech) with the 606 fragment as a probe, but this attempt was completely unsuccessful, and did not yield any positive clones.

Pursuant to this failure, an additional attempt to elongate the cDNA based on the initial sequence of the 606 fragment, by conducting 5′ Race reactions using Hela cell total mRNA as a template, also failed.

Although the inventors employed two independent methods, and conducted experiments according to routine methods several times, they were unsuccessful in identifying or isolating the full 606 gene.

Bioinformatic analysis was conducted in parallel to the above experiments, and the initial sequence of the 606 fragment was found to be in antisense orientation to a public cDNA, FLJ13287 (gi-10435245). Alignment of the initial fragment to the FLJ sequence was in reverse orientation to nucleotides 115-300 of the FLJ. The FLJ sequence predicts an open reading frame of 390 bp, from nucleotide 18 to nucleotide 407.

However, although the FLJ contains a complete open reading frame with a start codon and a stop codon, the inventors suspected that the sequence as published in the FLJ was only a portion of the gene, and that there is in effect a larger open reading frame; they proceeded to search for what they believed was the full gene.

Using BLAST with the FLJ13287 sequence as a query, an overlapping antisense EST (zb39c02) was found to extend the sequence information by 118 bp to the 5′ direction. The overlapping EST sequence represented the 354 bp of the 3′-end.

A database search revealed the 5′-end sequence of the same EST, which was 232 bp long. However, these end sequences of the EST did not align due to an un-sequenced gap. Therefore, primers were designed on both ends of the EST and a RT_PCR reaction was performed on mRNA extracted from is Hela cells.

The RT_PCR reactions performed failed as well, and this led the inventors to speculate that perhaps the 606 gene product is not expressed in many types of mammalian cells. Based on this idea, the inventors returned to cell-based experimentation, and attempted to identify a human cell line that expresses the product of the 606 gene. The basic principle driving the inventors was that Identification of a sample which contains expressed mRNA of the 606 gene could contribute to further experimentation which could lead, finally, to the isolation of the 606 gene. The inventors designed primers specific to the 606 gene, and RT_PCR reactions were performed on several human mRNA sources including Jurkat (T-cell line), U266B1 (plasmacytoma, myelomaBlymphocytes cell line), KCL22 and MOLT4. PCR products indicating the possible presence of mRNA of the 606 gene were obtained in sufficient quantities for further experimentation from RT_PCR performed on mRNA derived from U266B1 and Jurkat cells. These mRNAs were then used as the template in a RT_PCR reaction with primers designed to both ends of EST zb39c02 (for which extensive sequencing data was missing, as described above).

This reaction proved successful, and the resulting 774 bp product was analyzed. Sequences on both ends matched the ends of the EST zb39c02, and a gap of 188 bp which was missing from the EST data was identified by the inventors. Thus, the result was an extension of 719 bp to the 5′ direction of the 606 gene.

The consensus sequence of the FLJ13287 and extended sequence arrived at by the inventors was 2915 bp, and contained an open reading frame of 627 bp, which is significantly larger (by over 50%) then the original open reading frame of 390 bp present in the FLJ. However, this new open reading frame discovered by the inventors did not contain an ATG initiation codon, indicating that it was still not complete.

To complete the 5′ end sequence information, the inventors decided to perform a 5′-Race reaction on hela mRNA using reverse primers designed according to the 5′-end region of the consensus contig they had arrived at by combining the FLJ data with the additional EST data they had obtained through the experimentation described above. The reaction eventually yielded a sequence extension of 109 bp to the 5′ direction of the gene. This extension contained the desired ATG initiation codon, and an upstream 105 bp 5′-UTR region.

The full 606-cDNA sequence arrived at in this invention is 3024 bp, with a 1143 bp open reading frame. Thus, the original FLJ contains only 34% of the open reading frame of the polypeptide of this invention.

The initial cDNA fragment shown above aligns in reverse orientation to nucleotides 944-1127 of the final sequence.

Gene 606 has been shown to be a negative regulator of the pathway of Fas-induced apoptosis.

Analysis of the structure of the polypeptide 606 has shown that it has five WD40 domains. (See Garcia-Higuera, I et al, Biochemistry 1996, Nov 5, 34(44).)

The process described above by which the full gene was identified was a lengthy and complicated one, due to the failure of routine methods.

### Example 4

Screening Methods

The 606 gene identified herein can be used as a candidate gene in a screening assay for identifying and isolating compounds which modulate PCD, in particular, Fas-induced apoptosis. The compounds to be screened comprise inter alia small chemical molecules, antibodies, antisense oligonucleotides, antisense DNA or RNA molecules, proteins, polypeptides and peptides including peptido-mimetics and dominant negatives, and expression vectors. (A synthetic antisense oligonucleotide drug can inhibit translation of mRNA encoding the gene product of a Fas pathway gene.)

Many types of screening assays are known to those of ordinary skill in the art. The specific assay which is chosen depends to a great extent on the activity is of the candidate gene or the protein expressed thereby. Thus, if it is known that the expression product of a candidate gene has enzymatic activity, then an assay which is based on inhibition (or stimulation) of the enzymatic activity can be used. If the candidate protein is known to bind to a ligand or other interactor, then the assay can be based on the inhibition of such binding or interaction. When the candidate gene is a known gene, then many of its properties can also be known, and these can be used to determine the best screening assay. If the candidate gene is novel, then some analysis and/or experimentation is appropriate in order to determine the best assay to be used to find inhibitors of the activity of that candidate gene. The analysis can involve a sequence analysis to find domains in the sequence which shed light on its activity. Other experimentation described herein to identify the candidate gene and its activity can also be engaged in so as to identify the type of screen that is appropriate to find inhibitors or stimulators (enhancers), as the case can be, for the candidate gene or the protein encoded thereby.

As is well known in the art, the screening assays can be cell-based or non-cell-based. The cell-based assay is performed using eukaryotic cells and such cell-based systems are particularly relevant in order to directly measure the activity of candidate genes which are anti-apoptotic functional genes, i.e., expression of the gene prevents apoptosis or otherwise prevent cell death in target cells. One way of running such a cell-based assay uses tetracycline-inducible (Tet-inducible) gene expression. Tet-inducible gene expression is well known in the art; see for example, Hofmann et al, 1996, Proc Natl Acad Sci 93(11):5185-5190.

Tet-inducible retroviruses have been designed incorporating the Self-inactivating (SIN) feature of a 3′ Ltr enhancer/promoter retroviral deletion mutant. Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, thus indicating that expression is regulated uniformly within the infected cell population.

When dealing with candidate genes having anti-apoptotic function, such as gene 606, Tet-inducible expression prevents apoptosis in target cells. We screen for chemical compounds able to rescue the cells from the gene-triggered inhibition of apoptosis. (When dealing with candidate genes having pro-apoptotic function, Tet-inducible expression induces apoptosis in target cells. We screen for chemical compounds able to rescue the cells from the gene-triggered apoptosis.)

If the gene product of the candidate gene phosphorylates with a specific target protein, a specific reporter gene construct can be designed such that phosphorylation of this reporter gene product causes its activation, which can be followed by a color reaction. The candidate gene can be specifically induced, using the Tet-inducible system discussed above, and a comparison of induced versus non-induced genes provides a measure of reporter gene activation.

In a similar indirect assay, a reporter system can be designed that responds to changes in protein-protein interaction of the candidate protein. If the reporter responds to actual interaction with the candidate protein, a color reaction occurs.

One can also measure modulation of reporter gene activity by modulation of its expression levels via the specific candidate promoter or other regulatory elements. A specific promoter or regulatory element controlling the activity of a candidate gene is defined by methods well known in the art. A reporter gene is constructed which is controlled by the specific candidate gene promoter or regulatory elements. The DNA containing the specific promoter or regulatory agent is actually linked to the gene encoding the reporter. Reporter activity depends on specific activation of the promoter or regulatory element. Thus, inhibition or stimulation of the reporter is a direct assay of stimulation/inhibition of the reporter gene; see, for example, Komarov et al (1999), Science vol 285,1733-7 and Storz et al (1999) Analytical Biochemistry, 276, 97-104.

Various non-cell-based screening assays are also well within the skill of those of ordinary skill in the art. For example, if enzymatic activity is to be measured, such as if the candidate protein has a kinase activity, the target protein can be defined and specific phosphorylation of the target can be followed. The assay can involve either inhibition of target phosphorylation or stimulation of target phosphorylation, both types of assay being well known in the art; for example see Mohney et al (1998) J.Neuroscience,8 5285 and Tang et al (1997) J Clin. Invest. 100,1180 for measurement of kinase activity.

One can also measure in vitro interaction of a candidate polypeptide with interactors. In this screen, the candidate polypeptide is immobilized on beads. An interactor, such as a receptor ligand, is radioactively labeled and added. When it binds to the candidate polypeptide on the bead, the amount of radioactivity carried on the beads (due to interaction with the candidate polypeptide) can be measured. The assay indicates inhibition of the interaction by measuring the amount of radioactivity on the bead.

Any of the screening assays, according to the present invention, can include a step of identifying the chemical compound (as described above) which tests positive in the assay and can also include the further step of producing as a medicament that which has been so identified. It can also include steps of improving the chemical compound to increase its desired activity before incorporating the improved chemical compound into a medicament. It is considered that medicaments comprising such compounds are part of the present invention. The use of any such compounds identified for inhibition or stimulation of PCD, in particular, Fas-induced apoptosis, is also considered to be part of the present invention.

### Example 5

### Validation Data

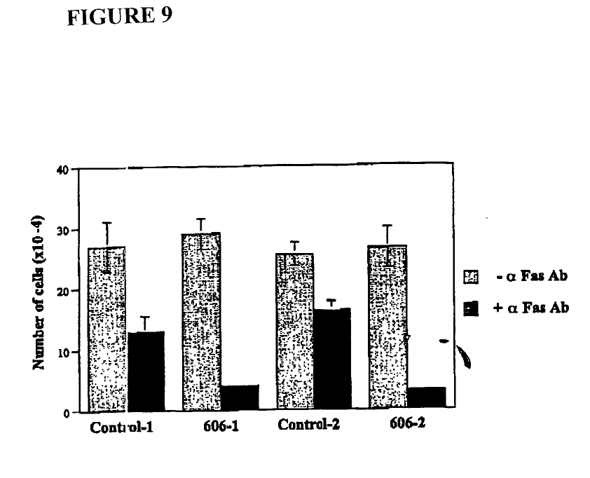

The antisense fragment of the 606 gene which was originally identified was inserted into the episomal vector used in the original study and transfected into Hela cells. Two independent and identical transfection experiments with the antisense fragment were performed. HeLa cells were transfected with either the anti-sense 606, or a control empty vector. 166,000 cells/well were plated in a 6 well plate and treated with 200 ng/ml anti-Fas Ab (Kamiya) the next day. 24 h after treatment, the live attached cells were counted by using trypan blue. The results are an average of duplicate wells for each pool, an can be seen in FIG. 9. As can be seen, the ratio of the percentage of live cells in the control to the percentage of live cells in the corresponding antisense transfected sample indicates 3.5 fold sensitization for 606-1 (the first transfection experiment) and 5.3 fold sensitization for 606-2. (the second transfection experiment).

This validaiton assay demonstrates that anti-sense fragments of the 606 gene sensitize cells to FAS mediated apoptosis as opposed to control cells; when the 606 gene is inhibited (by the antisense fragment) the cells are more sensitive to Fas induced apoptosis. Thus the 606 gene is a viability gene. The 606 gene may oppose general PCD genes or be involved in the induction of PCD by other stimuli.

### Example 6

### Manufacture and Use of the Claimed Products

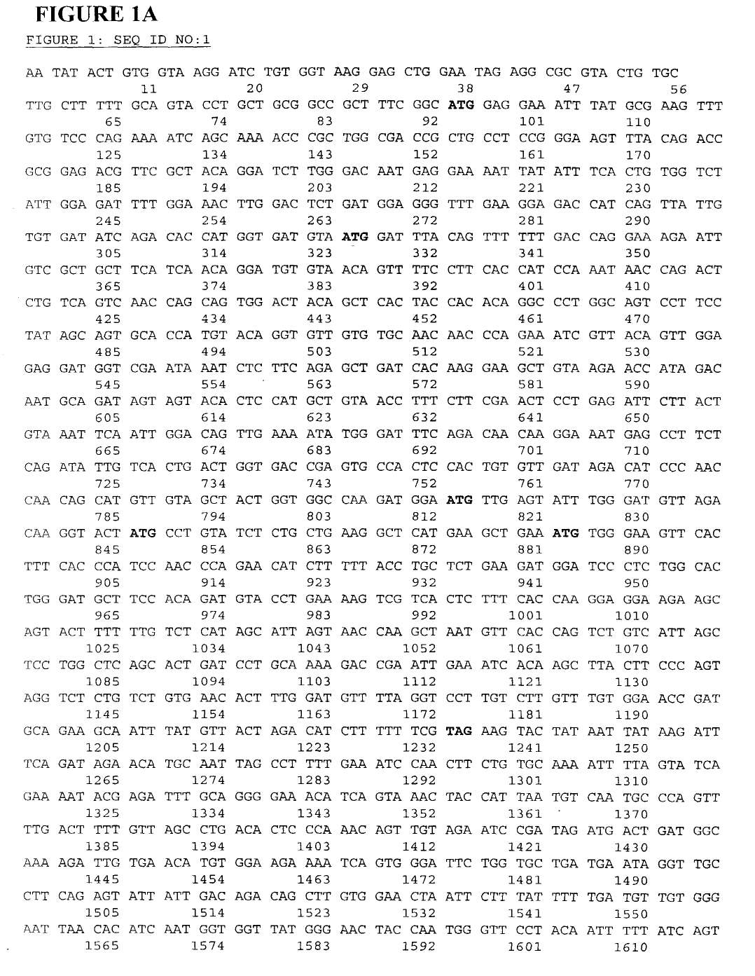

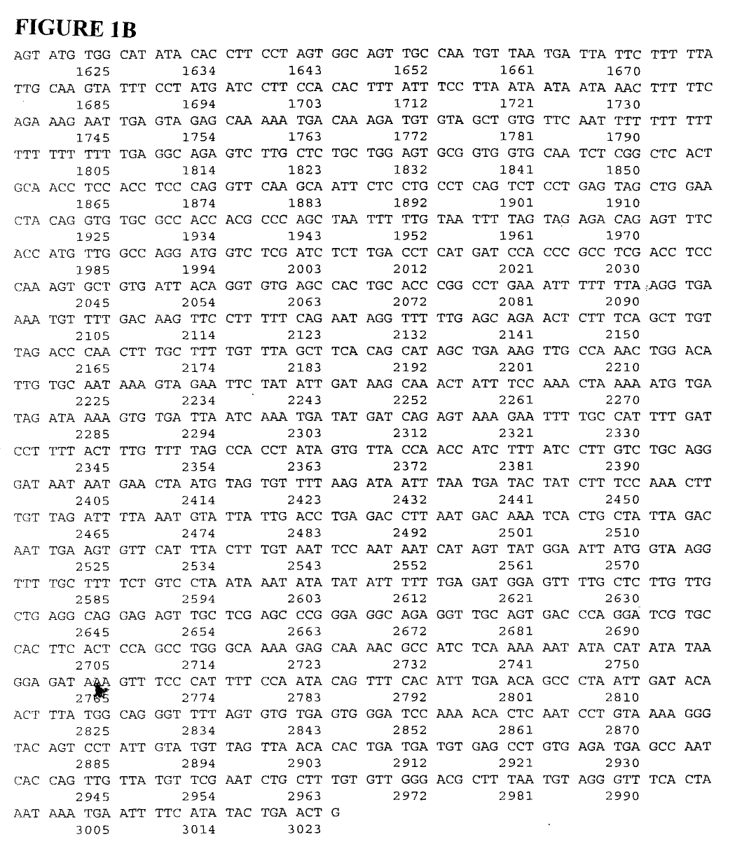

The claimed polynucleotides of the subject invention can be constructed by using a commercially available DNA synthesizing machine and SEQ ID NO 1 (the sequence shown in FIG. 1). For example, overlapping pairs of chemically synthesized fragments of the gene can be ligated using methods well known in the art (e.g. see U.S. Pat. No. 6,121,426).

Another means of isolating a claimed polynucleotide, e.g. the 606 gene, is to obtain a natural or artificially designed DNA fragment based on SEQ ID NO 1 (the sequence shown in FIG. 1). This DNA fragment is labeled by means of suitable labeling systems which are well known to those of skill in the art; see, e.g., Davis et al (1986). The fragment is then used as a probe to screen a lambda phage cDNA library or a plasmid cDNA library library using methods is well known in the art; see, generally, Sambrook et al, (1989) and Ausubel et al (1994-2000).

Colonies can be identified which contain clones related to the probe cDNA and these clones can be purified by known methods. The ends of the newly purified clones are then sequenced to identify full-length sequences and complete sequencing of full-length clones is performed by enzymatic digestion or primer walking. A similar screening and clone selection approach can be applied to clones from a genomic DNA library. The entire naturally-occurring cDNA or gene sequence, including any allelic variations thereof, all will have the same utility as discussed above for the identified polynucleotide. Alternatively, the claimed polynucleotides of the subject invention can be isolated by the polymerase chain reaction. Oligonucleotide primers can be designed based on SEQ ID NO 1 and the cDNA can be amplified from mRNA derived from a variety of biological sources by methods known in the art.

The claimed polynucleotides can be used inter alia as probes for diagnotic work. They can be used to diagnose cancerous cells, where the 606 gene is over-expressed and there are thus high levels of mRNA gene transcripts.

The claimed polypeptides can be produced by making synthetic peptides using a commercially available machine, and SEQ ID NO 2 (the sequence of FIG. 2.) Another means of making the claimed polypeptides is to clone cDNA or a fragment therof, using SEQ ID NO 1 (the sequence shown in FIG. 1), and express the resulting polypeptide, using methods known in the art.

The claimed polypeptides can be used for treatment as described in the above Examples. Additionally, the claimed polypeptides can be used for the manufacture of antibodies, which can be used to diagnose cancerous cells, where the 606 gene is over-expressed and there are thus high levels of 606 polypeptide. Furthermore, it is well-known that proteins and polypeptides are nutritious and edible.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

Agrawal, 1996. Antisense oligonucleotides: towards clinical trials, TIBTECH, 14:376.

Altschul et al. (1990) Basic local alignment search tool. J. Mol. Biol., 215:403410.

Askew, D. S., R. A. Ashmun, B. C. Simmons, and J. L. Cleveland. 1991. Constitutive c-myc expression in an IL-3-dependent myeloid cell line suppresses cell cycle arrest and accelerates apoptosis. Oncogene 6: 1915-1922.

Braun et al. (1996) Molecular and Cellular Biology, 15(8):4623-4630.

Calabretta, et al, 1996. Antisense strategies in the treatment of leukemias. Semin. Oncol. 23:78.

Chijiwa, T., M. Hagiwara, and H. Hidaka. 1989. A newly synthesized selective casein kinase I inhibitor, N-(2-aminoethyl)-5-chloroisoquinoline-8-sulfonamide, and affinity purification of casein kinase I from bovine testis. J Biol Chem 264: 4924-4927.

Cregg et al. (1993) Recent Advances in the Expression of Foreign Genes in Pichia pastoris. Bio/Technology 11:905-910.

Crooke, 1995. Progress in antisense therapeutics, Hematol. Pathol. 2:59.

Deiss and Kimchi (1991) A Genetic Tool Used to Identify Thioredoxin as a Mediator of a Growth Inhibitory Signal. Science252:117-120.

Deiss et al. (1995) Identification of a novel serine/threonine kinase and a novel 15-kD protein as potential mediators of the a interferon-induced cell death. Genes & Develop. 9:15-30.

Deiss et al. (1996) Cathepsin D protease mediates programmed cell death induced by interferon-ã, Fas/APO-1 and TNF-á. EMBO 15(15):3861-3870.

Delneste, Y., P. Jeannin, E. Sebille, J. P. Aubry, and J. Y. Bonnefoy. 1996. Thiols prevent Fas (CD95)-mediated T cell apoptosis by down-regulating membrane Fas expression. Eur J Immunol 26: 2981-2988.

Diatchenko et al. (1996) Proc. Natl. Acad. Sci.USA., 93:6025-6030.

Evan, G. I., A. H. Wyllie, C. S. Gilbert, T. D. Littlewood, H. Land, M. Brooks, C. M. Waters, L. Z. Penn, and D. C. Hancock. 1992. Induction of apoptosis in fibroblasts by c-myc protein. Cell 69: 119-128.

Felgner, 1997. Nonviral Strategeies for Gene Therapy. Scientific American. June, 1997, pgs 102-106.

Gewirtz, 1993. Oligodeoxynucleotide-based therapeutics for human leukemias, Stem Cells Dayt. 11:96.

Gilboa et al. (1986) Transfer and expression of cloned genes using retroviral vectors. BioTechniques 4(6):504-512.

Gubler and Hoffman (1983) Gene (Amst.) 25:263.

Gudkov et al. (1994) Cloning mammalian genes by expression selection of genetic suppressor elements: association of kinesin with drug resistance and cell immortalization. Proc. Natl. Acad. Sci. U.S.A., 91:3744-3748.

Hanania, et al 1995. Recent advances in the application of gene therapy to human disease. Am. J. Med. 99:537.

Hirt (1967) Selective extraction of polyoma DNA from infected mouse cell cultures. J. Mol. Biol., pp. 365-9.

Holzmayer et al. (1992) Isolation of dominant negative mutants and inhibitory antisense RNA sequences by expression selection of random DNA fragments. Nucleic Acids Res., 20:711-717.

Herskowitz I.: Functional inactivation of genes by dominant negative mutations. Nature. Sep. 17-23, 1987;329(6136):219-22. Review.

Izant and Weintraub (1984) Inhibition of thymidine kinase gene expression by anti-sense RNA: a molecular approach to genetic analysis. Cell, 36: 1007-1015.

Kissil et al. (1995) “Isolation of DAP3, a Novel Mediator of Interferon-ã-induced Cell Death” J. Biol. Chem. 270(46):27932-27936.

Lefebvre-d'Hellencourt et al, 1995. Immunomodulation by cytokine antisense oligonucleotides. Eur. Cytokine Netw. 6:7.

Lev-Lehman et al., 1997. Antisense Oligomers in vitro and in vivo. In Antisense Therapeutics, A. Cohen and S. Smicek, eds (Plenum Press, New York)

Levy-Strumpf et al. (1997) DAP-5, a novel homolog of eukaryotic translation initiation factor 4G isolated as a putative modulator of gamma interferon-induced programmed cell death. Mol. Cell. Biol., 17: 1615-1625.

Li and Cohen (1996) Tsg101: A novel tumor susceptibility gene isolated by controlled homozygous functional knockout of allelic loci in mammalian cells. Cell, 85:319-329.

Loke et al, 1989. Characterization of oligonucleotide transport into living cells. PNAS USA 86:3474.

Meissner et al. (1987) PNAS (USA) 84:4171.

Moi, P., K. Chan, I. Asunis, A. Cao, and Y. W. Kan. 1994. Isolation of NF-E2-related factor 2 (Nrf2), a NF-E2-like basic leucine zipper transcriptional activator that binds to the tandem NF-E2/AP1 repeat of the beta-globin locus control region. Proc Natl Acad Sci USA 91: 9926-9930.

Morgenstern, J. P. and H. Land. 1990. A series of mammalian expression vectors and characterization of their expression of a reporter gene in stably and transiently transfected cells. Nucleic Acids Res 18: 1068.

Morrow, C. S., P. K. Smitherman, and A. J. Townsend. 2000. Role of multidrug-resistance protein 2 in glutathione S-transferase P1-1-mediated resistance to 4-nitroquinoline 1-oxide toxicities in HepG2 cells [In Process Citation]. Mol Carcinog 29: 170-178.

Nagata, S. 1997. Apoptosis by death factor. Cell 88: 355-365.

Ohtsubo, T., S. Kamada, T. Mikami, H. Murakami, and Y. Tsujimoto. 1999. Identification of NRF2, a member of the NF-E2 family of transcription factors, as a substrate for caspase-3(-like) proteases. Cell Death Differ 6: 865-872.

Radhakrishnan et al., 1990. The automated synthesis of sulfur-containing oligodeoxyribonucleotides using 3H-1,2-Benzodithiol-3-0ne 1,1 Dioxide as a sulfur-transfer reagent. J. Org. Chem. 55:4693-4699.

Roninson et al. (1995) Genetic suppressor elements: new tools for molecular oncology-thirteenth Cornelius P. Rhoads Memorial Award Lecture. Cancer Res., 55:4023-4028.

Schena et al., 1995, Aiello et al., 1994, Shen et al., 1995, Bauer et al., 1993, Liang and Pardee, 1992, Liang and Pardee, 1995, Liang et al., 1993, Braun et al., 1995, Hubank and Schatz, 1994

Schwarze, S. R., A. Ho, A. Vocero-Akbani, and S. F. Dowdy. 1999. In vivo protein transduction: delivery of a biologically active protein into the mouse [see comments]. Science 285: 1569-1572.

Shi, Y., J. M. Glynn, L. J. Guilbert, T. G. Cotter, R. P. Bissonnette, and D. R. Green. 1992. Role for c-myc in activation-induced apoptotic cell death in T cell hybridomas. Science 257: 212-214.

Shoemaker, D. D., D. A. Lashkari, D. Morris, M. Mittmann, and R. W. Davis. 1996. Quantitative phenotypic analysis of yeast deletion mutants using a highly parallel molecular bar-coding strategy [see comments]. Nat Genet 14: 450-456.

Smith et al. (1995) genetic footprinting: a genomic strategy for determining a gene's function given its sequence. PNAS 92:6479-6489.

Smith, V., K. N. Chou, D. Lashkari, D. Botstein, and P. O. Brown. 1996. Functional analysis of the genes of yeast chromosome V by genetic footprinting. Science 274: 2069-2074.

Spanaus, K. S., R. Schlapbach, and A. Fontana. 1998. TNF-alpha and IFNgamma render microglia sensitive to Fas ligand-induced apoptosis by induction of Fas expression and down-regulation of Bcl-2 and Bcl-xL. Eur J Immunol 28: 4398-4408.

Su et al. (1993) LYAR, a novel nucleolar protein with zinc finger DNA-binding motifs, is involved in cell growth regulation. Genes Dev., 7:735-748.Schena et al. (1995) Science, 270:467-470.

Wagner et al., 1996. Potent and selective inhibition of gene expression by an antisense heptanucleotide. Nature Biotechnology 14:840-844.

Wagner, 1994. Gene inhibition using antisense oligodeoxynucleotides. Nature 372:333.

Wagner, A. J., J. M. Kokontis, and N. Hay. 1994. Myc-mediated apoptosis requires wild-type p53 in a manner independent of cell cycle arrest and the ability of p53 to induce p21waf1/cip1. Genes Dev 8: 2817-2830.

Watson, R. W., O. D. Rotstein, M. Jimenez, J. Parodo, and J. C. Marshall. 1997. Augmented intracellular glutathione inhibits Fas-triggered apoptosis of activated human neutrophils Blood 89:4175-4181.

We claim:

1. A purified polypeptide comprising consecutive amino acids the sequence of which extends from position 242 through position 380 of SEQ ID No:2.

2. The polypeptide of claim 1 wherein the polypeptide comprises consecutive amino acids the sequence of which extends from position 241 through position 380 of SEQ ID No:2

3. The polypeptide of claim 1 wherein the polypeptide comprises consecutive amino acids the sequence of which extends from position 79 through position 380 of SEQ ID No:2.

4. The polypeptide of claim 1 wherein the polypeptide comprises consecutive amino acids the sequence of which extends from position 78 through position 380 of SEQ ID No:2

5. The polypeptide of claim 1 wherein the polypeptide comprises consecutive amino acids the sequence of which extends from position 2 through position 380 of SEQ ID No:2

6. The polypeptide of claim 1 wherein the polypeptide comprises consecutive amino acids the sequence of which extends from position 1 through position 380 of SEQ ID No:2.

7. A purified polypeptide encoded by a polynucleotide having at least 30, preferably at least 50 more preferably at least 70, most preferably at 30 least 100 consecutive nucleotides from position 1 to position 108 of SEQ ID NO:1.

8. A purified polypeptide encoded by a polynucleotide having at least 30, preferably at least 50 more preferably at least 70, even more preferably at least 100, even more preferably at least 150, most preferably at least 200 consecutive nucleotides from position 340 to position 831 of SEQ ID NO:1.

9. The polypeptide of any one of claims 1-8 wherein the polypeptide is encoded by consecutive nucleotides present in a plasmid designated pMLPD-606 Bac1, deposited under ATCC deposit No. PTA-4348.

10. A purified polypeptide according to claim 5 having the biological activity of opposing Fas-mediated apoptosis.

11. A purified polypeptide having at least 70% homology to, and retaining the biological activity of the polypeptide of claim 10.

12. A purified polypeptide according to claim 1 having the inhibitory activity of a dominant negative peptide to the polypeptide of claim 10 or 11.

13. A purified polypeptide having at least 70% homology to, and retaining the inhibitory activity of the polypeptide of claim 12.

14. A pharmaceutical composition comprising the polypeptide according to any one of claims 1-13.

15. An antibody directed to an epitope on a polypeptide according to any one of claims 1-13.

16. An antibody binding specifically to the polypeptide of claims 1-13.

17. An isolated polynucleotide comprising consecutive nucleotides having a sequence as set forth in SEQ ID NO:1 and homologs or complements thereof.

18. An isolated polynucleotide comprising consecutive nucleotides having a sequence as set forth from position 93 through position 1232 of SEQ ID NO:1 and homologs or complements thereof or comprising consecutive nucleotides having a sequence incorporated in a plasmid designated pMLPD-606 Bac1, deposited under ATCC deposit No. PTA-4348, and homologs or complements thereof.

19. An isolated polynucleotide which encodes the polypeptide of any one of claims 1-13.

20. An isolated polynucleotide which encodes the polypeptide of any one of claims 1-13 comprising a strand of a full-length cDNA.

21. An antisense polynucleotide complementary to the polynucleotide according to any one of claims 17-20.

22. A pharmaceutical composition comprising the isolated polynucleotide according to any one of claims 17-21.

23. A vector comprising the isolated polynucleotide according to any one of claims 17-21.

24. A pharmaceutical composition comprising the vector according to claim 23.

25. A method of treating a tumor or an auto-immune disease in a subject which comprises administering to the subject a therapeutically effective amount of a chemical composition which inhibits the biological activity of the polypeptide of any one of claims 1-11.

26. The method according to claim 25 wherein the chemical composition comprises a polynucleotide according to claim 21, or a vector comprising a polynucleotide according to claim 21.

27. The method according to claim 25 wherein the chemical composition comprises a polypeptide according to claim 12 or 13.

28. A method of treating degenerative disease in a subject which comprises administering to the subject a therapeutically effective amount of a chemical composition which enhances or stimulates the biological activity of the polypeptide of claims 1-11.

29. The method according to claim 28 wherein the chemical composition comprises a polynucleotide according to any one of claims 17-20, or a vector comprising a polynucleotide according to one of claims 17-20.

30. The method according to claim 28 wherein the chemical composition comprises a polypeptide according to any one of claims 1-11.

31. Use of the polypeptide of any one of claims 1-13 or the polynucleotide of any one of claims 17-21 or the or the antibody of claims 15-16 in the preparation of a medicament.

32. A method for identifying a chemical compound which modulates Fas-mediated apoptosis which comprises:
(a) contacting a cell expressing the polypeptide of any one of claims 1-13 with the compound; and
(b) determining the ability of the compound to modulate Fas-mediated apoptosis of the cell as compared to a control.

33. The method according to claim 32, wherein the cell in the contacting step has been genetically engineered to express the polypeptide.

34. A method of preparing a pharmaceutical composition which comprises:
(a) identifying a chemical compound which modulates Fas-mediated apoptosis according to the method of claim 32, and;
(b) admixing said compound or a chemical homolog or analog thereof with a pharmaceutically acceptable carrier.

35. A method of screening a plurality of chemical compounds not known to modulate Fas-mediated apoptosis to identify a compound which modulates Fas-mediated apoptosis which comprises:
(a) contacting cells expressing the polypeptide of any one of claims 1-13 with the plurality of chemical compounds not known to modulate Fas-mediated apoptosis;
(b) determining whether the Fas-mediated apoptosis of the cell is modulated in the presence of the compounds, as compared to a control; and if so
(c) separately determining whether the modulation of Fas-mediated apoptosis is increased by each compound included in the plurality of compounds, so as to thereby identify the compound which modulates Fas-mediated apoptosis.

36. The method according to claim 34, wherein the cell in the contacting step has genetically engineered to express the polypeptide.

37. A method of preparing a pharmaceutical composition which comprises:
(a) identifying a chemical compound which modulates Fas-mediated apoptosis according to the method of claim 35, and;
(b) admixing said compound or a chemical homolog or analog thereof with a pharmaceutically acceptable carrier.

38. A non cell-based method for identifying a compound which modulates Fas-mediated apoptosis comprising:
(a) measuring the interaction of the polypeptide of any one of claims 1-13 or the polynucleotide of any one of claims 17-21 to an interactor;
(b) contacting the polypeptide of any one of claims 1-13 or the polynucleotide of any one of claims 17-21 with said compound; and
(c) determining whether the interaction between the polypeptide of any one of claims 1-13 or the polynucleotide of any one of claims 17-21 and said interactor is affected by said compound.

39. The method according to claim 38 wherein the interaction of step (a) occurs through a WD40 domain on the polypeptide or the polynucleotide.

40. A method of preparing a pharmaceutical composition which comprises:
(a) identifying a chemical compound which modulates Fas-mediated apoptosis according to the method of claim 38, and;
(b) admixing said compound or a chemical homolog or analog thereof with a pharmaceutically acceptable carrier.

41. A kit for identifying a compound which modulates Fas-mediated apoptosis in a cell comprising:
(a) the polypeptide of any one of claims 1-13 or the polynucleotide of any one of claims 17-21; and
(b) an interactor with which the polypeptide of any one of claims 1-13 or the polynucleotide of any one of claims 17-21 interacts;
(c) means for measuring the interaction of the polypeptide of any one of claims 1-13 or the polynucleotide of any one of claims 17-21 with the interactor;
(d) means of contacting the polypeptide of any one of claims 1-13 or the polynucleotide of any one of claims 17-21 with said compound; and
(e) means of determining whether the interaction between the polypeptide of any one of claims 1-13 or the polynucleotide of any one of claims 17-21 and the interactor is affected by said compound.
